(19) United States
(12) Patent Application Publication
(10) Pub. No.: US 2026/0269063 A1
(43) Pub. Date: Sep. 10, 2026

(54) CONSTRAINT-GOVERNED NEURO-SYMBOLIC REASONING STRUCTURE WITH MULTI-FRAMEWORK EVALUATION FOR DEVICE OUTPUT GENERATION

(71) Applicant: WONDER SCIENCES, INC., Marina del Rey, CA (US)

(72) Inventor: Ryan R. Magnussen, Marina del Rey, CA (US)

(21) Appl. No.: 19/558,281

(22) Filed: Mar. 5, 2026

Related U.S. Application Data
(60) Provisional application No. 63/768,813, filed on Mar. 7, 2025.

(51) Int. Cl. G16H 50/20; G16H 10/60

(52) U.S. Cl. CPC G16H 50/20; G16H 10/60

(57) ABSTRACT

A system and a method are disclosed for constraint-governed neuro-symbolic reasoning with multi-framework evaluation. The system accesses user input and biometric signals. The system performs parallel feature extraction on the user input and the biometric signals to extract features for each signal stream, wherein the features are concatenated into a feature vector representing intent of the user. The system accesses a hierarchical universal knowledge graph comprising nodes representing source material, nodes representing concepts, claims, frameworks, or rules derived from the source material, and edges weighting connections between nodes. The system generates candidate prompts based on the feature vector of the intent of the user and the hierarchical universal knowledge graph. The system executes each candidate prompt on a language model to output candidate responses to the candidate prompts, with a reasoning trace for each candidate response. The system performs validation of each candidate response by computing evaluation score(s) based on the candidate response and the reasoning trace. The system selects one candidate response based on the evaluation scores. The system transmits the selected response for presentation.

### CROSS-REFERENCE TO RELATED APPLICATIONS

The present application claims the benefit of and priority to U.S. Provisional Application No. 63/768,813 filed on Mar. 7, 2025, which is incorporated by reference.

### BACKGROUND

Contemporary artificial intelligence systems, including large-scale language models and related generative architectures, predominantly rely on probabilistic inference over high-dimensional parameter spaces to generate outputs responsive to user inputs. While such systems may produce linguistically fluent responses, they typically lack structured reasoning controls and formal governance mechanisms embedded within the inference architecture itself.

Existing systems frequently exhibit one or more of the following technical deficiencies: absence of explicit knowledge grounding, non-deterministic reasoning behavior, non-executable governance controls, lack of cross-framework evaluation, no structured conflict detection or reconciliation, absence of uncertainty propagation, limited auditability and reproducibility, unbounded personalization mechanisms.

Existing safety and alignment techniques are commonly implemented as external moderation layers, e.g., static rule lists, classifier-based gating systems, or output post-processors. These approaches operate adjacent to, rather than within, the reasoning architecture and therefore do not provide deterministic, enforceable governance during response generation, evaluation, and selection.

Accordingly, there exists a need for an improved computer-implemented reasoning infrastructure that architecturally separates candidate generation from structured validation, integrates enforceable governance rules as executable constraint logic, performs multi-framework evaluation of candidate outputs, detects, quantifies, and reconciles cross-framework conflict, propagates uncertainty metrics derived from structured reasoning signals, produces auditable, machine-readable output packets, and supports longitudinal personalization subject to explicit consent and retention controls.

### SUMMARY

According to an aspect of the invention, a computer-implemented reasoning infrastructure system comprises one or more processors and one or more non-transitory computer-readable media storing instructions that, when executed by the one or more processors, cause the system to receive an input query and contextual data, generate a normalized intent representation from the input query, retrieve from a hierarchical universal knowledge graph (HUKG) a structured subgraph responsive to the normalized intent representation, the HUKG comprising typed nodes and typed edges each associated with provenance metadata, generate a plurality of candidate reasoning outputs conditioned on the structured subgraph, evaluate each candidate reasoning output using a plurality of distinct framework evaluators each configured to generate at least a score, a structured rationale, and one or more constraint signals, apply a constraint execution engine configured to enforce governance rules expressed as executable condition-action logic for performing a remedial action (e.g., one or more hard constraints capable of blocking, repairing, redirecting, or escalating a candidate reasoning output), compute a conflict metric quantifying divergence among outputs of the plurality of framework evaluators, compute an uncertainty representation based at least in part on retrieval coverage, the conflict metric, weighted rule violations, and model variance, select a validated reasoning output based on the framework evaluator outputs, the conflict metric, and the uncertainty representation, generate a structured output packet comprising answer content, a machine-readable explanation trace linking portions of the answer content to nodes of the HUKG, provenance references, the uncertainty representation, and applied framework identifiers, and generate and store an audit record comprising a hash of the normalized intent representation, model version identifiers, policy and rule version identifiers, and a hash of the structured output packet. This architecture materially improves reliability and consistency of reasoning outputs by embedding governance enforcement, conflict quantification, and uncertainty propagation within the reasoning architecture itself, thereby enabling deterministic enforcement of safety and policy constraints and providing cross-framework transparency with reproducible and auditable outputs suitable for regulated and safety-sensitive domains.
Clause 1. A computer-implemented method comprising: accessing user input, provided via an input device on a client device and biometric signals captured by one or more health sensors; performing parallel feature extraction on the user input and the biometric signals to extract features for each signal stream, wherein the features are concatenated into a feature vector representing intent of the user; accessing a hierarchical universal knowledge graph comprising a first plurality of nodes representing source material, a second plurality of nodes representing concepts, claims, frameworks, or rules derived from the source material, and a plurality of edges weighting connections between nodes; generating a plurality of candidate prompts based on the feature vector of the intent of the user and the hierarchical universal knowledge graph; executing each candidate prompt on a language model to output a plurality of candidate responses to the candidate prompts, wherein the language model is further configured to output a reasoning trace for each candidate response tracing contribution of a subset of nodes in the hierarchical universal graph; performing validation of each candidate response by computing one or more evaluation scores based on the candidate response and the reasoning trace; selecting one of the candidate responses based on the evaluation scores; and transmitting the selected response to the client device for presentation on an output device of the client device.
Clause 2. The computer-implemented method of clause 1, wherein accessing the hierarchical universal knowledge graph comprises: retrieving a structured subgraph responsive to the feature vector, wherein the structured subgraph comprises a subset of the first plurality of nodes and the second plurality of nodes connected by a subset of the plurality of edges.
Clause 3. The computer-implemented method of clause 1, wherein the biometric signals comprise one or more of: heart rate variability signals, respiratory cadence signals, electroencephalography signals, galvanic skin response signals, motion signals, posture signals, or interaction telemetry signals.
Clause 4. The computer-implemented method of clause 1, wherein performing parallel feature extraction comprises: applying a first feature extractor to the user input to extract a first set of features; applying a second feature extractor to the biometric signals to generate a second set of features; and concatenating the first set of features and the second set of features to generate the feature vector representing the intent of the user.
Clause 5. The computer-implemented method of clause 1, wherein the second plurality of nodes comprises framework nodes representing reasoning frameworks, and wherein performing validation comprises applying a plurality of framework evaluators corresponding to the framework nodes to generate the one or more evaluation scores.
Clause 6. The computer-implemented method of clause 5, wherein performing validation further comprises: computing a conflict score quantifying divergence among the one or more evaluation scores generated by different framework evaluators; and computing an uncertainty score based on retrieval coverage, the conflict score, weighted rule violations, or model variance, or some combination thereof.
Clause 7. The computer-implemented method of clause 1, further comprising: applying a constraint execution engine to each candidate response to enforce governance rules expressed as executable condition-action logic; and performing a remedial action to one or more of the candidate responses based on the governance rules.
Clause 8. The computer-implemented method of clause 1, further comprising: generating a structured output packet comprising the selected response and a machine-readable explanation trace linking portions of the selected response to nodes of the hierarchical universal knowledge graph, wherein transmitting the selected response comprises transmitting the structured output packet to the client device for presentation of the selected response.
Clause 9. The computer-implemented method of clause 1, further comprising: accessing a longitudinal reasoning profile associated with the user, wherein the longitudinal reasoning profile comprises framework weighting preferences, governance constraint preferences, explanation depth settings, retention configuration, or deletion controls, or some combination thereof; and modulating generation of the plurality of candidate prompts based on the longitudinal reasoning profile.
Clause 10. The computer-implemented method of clause 9, further comprising: receiving user feedback to presentation of the selected response; scoring the selected response and the reasoning trace based on the user feedback; and modifying the longitudinal reasoning profile based on the score.
Clause 11. A non-transitory computer-readable storage medium storing instructions that, when executed, cause a computer processor to perform operations comprising: accessing user input, provided via an input device on a client device and biometric signals captured by one or more health sensors; performing parallel feature extraction on the user input and the biometric signals to extract features for each signal stream, wherein the features are concatenated into a feature vector representing intent of the user; accessing a hierarchical universal knowledge graph comprising a first plurality of nodes representing source material, a second plurality of nodes representing concepts, claims, frameworks, or rules derived from the source material, and a plurality of edges weighting connections between nodes; generating a plurality of candidate prompts based on the feature vector of the intent of the user and the hierarchical universal knowledge graph; executing each candidate prompt on a language model to output a plurality of candidate responses to the candidate prompts, wherein the language model is further configured to output a reasoning trace for each candidate response tracing contribution of a subset of nodes in the hierarchical universal graph; performing validation of each candidate response by computing one or more evaluation scores based on the candidate response and the reasoning trace; selecting one of the candidate responses based on the evaluation scores; and transmitting the selected response to the client device for presentation on an output device of the client device.
Clause 12. The non-transitory computer-readable storage medium of clause 11, wherein accessing the hierarchical universal knowledge graph comprises: retrieving a structured subgraph responsive to the feature vector, wherein the structured subgraph comprises a subset of the first plurality of nodes and the second plurality of nodes connected by a subset of the plurality of edges.
Clause 13. The non-transitory computer-readable storage medium of clause 11, wherein the biometric signals comprise one or more of: heart rate variability signals, respiratory cadence signals, electroencephalography signals, galvanic skin response signals, motion signals, posture signals, or interaction telemetry signals.
Clause 14. The non-transitory computer-readable storage medium of clause 11, wherein performing parallel feature extraction comprises: applying a first feature extractor to the user input to extract a first set of features; applying a second feature extractor to the biometric signals to generate a second set of features; and concatenating the first set of features and the second set of features to generate the feature vector representing the intent of the user.
Clause 15. The non-transitory computer-readable storage medium of clause 11, wherein the second plurality of nodes comprises framework nodes representing reasoning frameworks, and wherein performing validation comprises applying a plurality of framework evaluators corresponding to the framework nodes to generate the one or more evaluation scores.
Clause 16. The non-transitory computer-readable storage medium of clause 15, wherein performing validation further comprises: computing a conflict score quantifying divergence among the one or more evaluation scores generated by different framework evaluators; and computing an uncertainty score based on retrieval coverage, the conflict score, weighted rule violations, or model variance, or some combination thereof.
Clause 17. The non-transitory computer-readable storage medium of clause 11, the operations further comprising: applying a constraint execution engine to each candidate response to enforce governance rules expressed as executable condition-action logic; and performing a remedial action to one or more of the candidate responses based on the governance rules.
Clause 18. The non-transitory computer-readable storage medium of clause 11, the operations further comprising: generating a structured output packet comprising the selected response and a machine-readable explanation trace linking portions of the selected response to nodes of the hierarchical universal knowledge graph, wherein transmitting the selected response comprises transmitting the structured output packet to the client device for presentation of the selected response.
Clause 19. The non-transitory computer-readable storage medium of clause 11, the operations further comprising: accessing a longitudinal reasoning profile associated with the user, wherein the longitudinal reasoning profile comprises framework weighting preferences, governance constraint preferences, explanation depth settings, retention configuration, or deletion controls, or some combination thereof; and modulating generation of the plurality of candidate prompts based on the longitudinal reasoning profile.
Clause 20. The non-transitory computer-readable storage medium of clause 19, the operations further comprising: receiving user feedback to presentation of the selected response; scoring the selected response and the reasoning trace based on the user feedback; and modifying the longitudinal reasoning profile based on the score.
Clause 21. A system comprising: a computer processor; and a non-transitory computer-readable storage medium storing instructions that, when executed by the computer processor, cause the computer processor to perform operations comprising: accessing user input, provided via an input device on a client device and biometric signals captured by one or more health sensors; performing parallel feature extraction on the user input and the biometric signals to extract features for each signal stream, wherein the features are concatenated into a feature vector representing intent of the user; accessing a hierarchical universal knowledge graph comprising a first plurality of nodes representing source material, a second plurality of nodes representing concepts, claims, frameworks, or rules derived from the source material, and a plurality of edges weighting connections between nodes; generating a plurality of candidate prompts based on the feature vector of the intent of the user and the hierarchical universal knowledge graph; executing each candidate prompt on a language model to output a plurality of candidate responses to the candidate prompts, wherein the language model is further configured to output a reasoning trace for each candidate response tracing contribution of a subset of nodes in the hierarchical universal graph; performing validation of each candidate response by computing one or more evaluation scores based on the candidate response and the reasoning trace; selecting one of the candidate responses based on the evaluation scores; and transmitting the selected response to the client device for presentation on an output device of the client device.

### BRIEF DESCRIPTION OF THE DRAWINGS

FIG. 1 illustrates a representative operating environment in which a constraint-governed neuro-symbolic reasoning engine operates, according to some embodiments.

FIG. 2 illustrates a block diagram of the analytics system, according to some embodiments.

FIG. 3A illustrates the hierarchical layered structure of a knowledge graph, according to some embodiments.

FIG. 3B illustrates the knowledge graph structure comprising nodes, edges, and a metadata layer, according to some embodiments.

FIG. 4 illustrates an illustrative workflow of the reasoning infrastructure deployed by the analytics system, according to some embodiments.

FIG. 5 illustrates a workflow of an intent decoding module, according to some embodiments.

FIG. 6A illustrates a workflow of a response validation module that processes candidate responses and reasoning traces through multiple evaluator nodes to generate evaluation scores, according to some embodiments.

FIG. 6B illustrates a workflow of an aggregator that computes a conflict score for validation of a candidate response, according to some embodiments.

FIG. 7 illustrates a workflow of a response validation module that computes an uncertainty score for validation of a candidate response, according to some embodiments.

FIG. 8 illustrates a workflow of a user interface module that processes candidate responses and reasoning traces through a hashing layer to generate an output hash, according to some embodiments.

FIG. 9 illustrates a workflow of a longitudinal profiling module to curate a longitudinal profile for a user, e.g., over many sessions with the reasoning engine, according to some embodiments.

FIG. 10 is a method flowchart describing constraint-governed neuro-symbolic reasoning, according to some embodiments.

FIG. 11 shows a block diagram of a neural network model, according to one or more embodiments.

FIG. 12 is a conceptual diagram of functional blocks of a transformer-based neural network model 1200, according to one or more embodiments.

The figures and flowcharts presented herein are illustrative of representative embodiments and are not intended to limit the scope of the invention to the specific configurations, arrangements, or sequences depicted. A person having ordinary skill in the art, upon review of the present disclosure, would readily understand the underlying principles governing the illustrated architectures and would be capable of implementing variations, modifications, and alternative embodiments that achieve the same functional results without departing from the scope of the invention as defined by the appended claims.

### DETAILED DESCRIPTION

### Overview

The present disclosure provides systems, methods, and non-transitory computer-readable media for a constraint-governed, neuro-symbolic reasoning infrastructure platform configured to generate, evaluate, validate, and audit structured reasoning outputs under executable governance controls.

In contrast to conventional probabilistic language generation systems, the disclosed architecture separates candidate generation from structured validation and embeds enforceable governance directly within the reasoning pipeline.

The reasoning infrastructure operates as a layered, constraint-executable pipeline that separates probabilistic generation from deterministic validation and governance enforcement.

By embedding governance enforcement, structured evaluation, conflict quantification, and uncertainty propagation directly within the reasoning pipeline, the disclosed architecture provides material technical improvements over conventional probabilistic language generation systems.

### System and Networking Environment

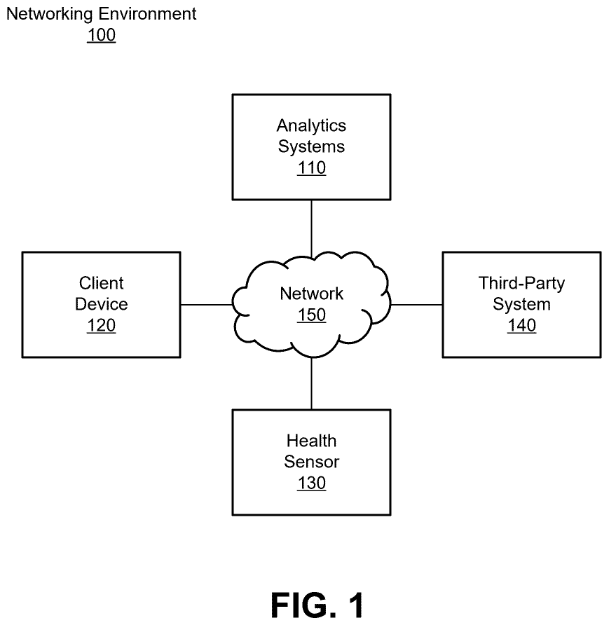

FIG. 1 illustrates a representative operating environment in which a constraint-governed neuro-symbolic reasoning engine communicates with one or more user devices, enterprise systems, external data sources, policy repositories, and audit logging subsystems over a network. The reasoning engine may be deployed in cloud-based server environments, private enterprise deployments, federated or hybrid distributed architectures, on-device inference systems, edge-computing environments, or regulatory or compliance-integrated systems.

The reasoning engine may communicate with user devices, enterprise data systems, external structured data sources, model registries and policy versioning services, and regulatory audit systems through wireless, wired, or a combination of wireless and wired communication technologies. The analytics system is configured with a processor and non-transitory computer-readable storage medium storing computer instructions that, when executed by the processor, cause the processor to process sequence reads or to perform one or more steps of the methods or processes disclosed herein. The reasoning engine may operate in centralized, distributed, hybrid, cloud-based, on-device, or federated configurations.

As shown in FIG. 1, the networking environment 100 includes an analytics system 110, a client device 120, one or more health sensors 130, and a third-party system 140 communicating over a network 150.

The analytics system 110 performs computational analyses with the reasoning engine to provide outputs to the client device 120. The analytics system 110 receives signal streams from the client device 120, the health sensor 130, the third-party system 140, or some combination thereof. The analytics system 110 applies the reasoning engine to the received signal streams to determine an output based on the normalized intent representation, retrieved knowledge subgraphs, multi-framework evaluation, constraint enforcement, conflict quantification, uncertainty propagation, or some combination thereof. The analytics system 110 provides the output to the client device 120, and may also provide outputs or audit records to the third-party system 140 or other connected components in the networking environment 100.

The client device 120 presents a user interface for the user to communicate with the analytics system 110. The client device 120 may be a mobile device, a wearable device, another computing system, or some combination thereof. The client device 120 may include one or more input devices, one or more output devices, or some combination thereof. For example, the input devices may include a touchscreen, a text input window, a microphone, other mechanical inputs, other audio inputs, or some combination thereof. For example, the output devices may include an electronic display, a speaker, a haptic assembly, or some combination thereof. As the client device 120 receives data from the analytics system 110, the client device 120 may output the data for presentation to the user via the one or more output devices.

The health sensor 130 captures biometric signals characterizing a user's health state. In one or more embodiments, the health sensor 130 may be implemented on the client device 120. The health sensor may capture biometric signals in conjunction with user input via the client device 120.

In one or more embodiments, the health sensor 130 captures heart rate or derivative signals. The health sensor 130 may include one or more electrodes for measuring heart-related signals, including electrocardiogram (EKG) signals, heart rate, heart rate variability, or other cardiac metrics.

In one or more embodiments, the health sensor 130 captures respiratory cadence signals. The health sensor 130 may include one or more sensors for measuring respiratory patterns, including breath cycle duration, inhale-exhale symmetry, respiratory coherence, or other respiratory metrics.

In one or more embodiments, the health sensor 130 captures electroencephalography (EEG) signals. The health sensor 130 may include one or more electrodes for measuring neural oscillation features, including alpha, theta, beta, or gamma frequency bands, neural synchrony patterns, or other brain activity metrics.

In one or more embodiments, the health sensor 130 captures galvanic skin response (GSR) signals. The health sensor 130 may include one or more sensors for measuring electrodermal activity, skin conductance, or other autonomic arousal indicators.

In one or more embodiments, the health sensor 130 captures motion or posture signals. The health sensor 130 may include one or more accelerometers, gyroscopes, or other motion sensors for measuring body position, movement patterns, activity levels, or other kinematic metrics.

In one or more embodiments, the health sensor 130 captures interaction telemetry signals. The health sensor 130 may include one or more sensors for measuring interaction latency, focus consistency, scrolling pauses, typing cadence, hesitation intervals, or other behavioral micro-patterns.

The third-party system 140 provides external data sources, policy repositories, or audit logging services to the analytics system 110. The third-party system 140 may include enterprise knowledge repositories, regulatory compliance databases, external structured data sources, or other systems that provide information or services to support the reasoning infrastructure.

The architecture supports distributed execution without compromising governance enforcement or traceability. The system maintains deterministic governance enforcement and audit-grade traceability across all deployment configurations, whether centralized or distributed. The modular architecture enables dynamic replacement of individual components, version updates to models or policies, and expansion of framework evaluators without requiring changes to the overall system architecture or compromising the integrity of governance controls.

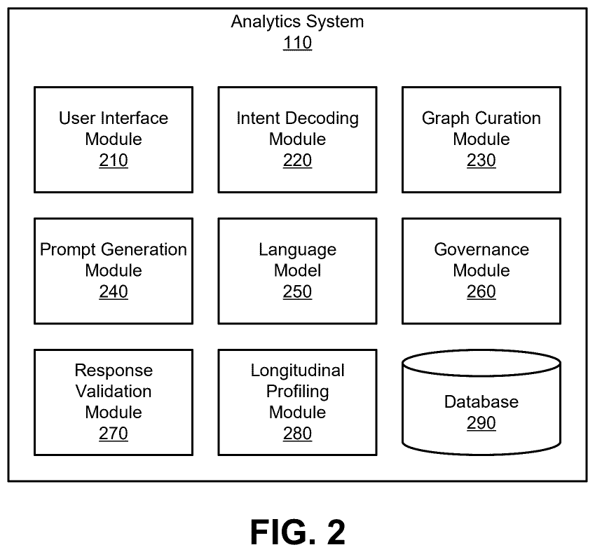

FIG. 2 illustrates a block diagram of the analytics system 110, according to some embodiments. The analytics system 110 includes one or more modules for processing inputs from the client device 110, to generate outputs responsive to the inputs. The analytics system 110 includes, for example, a user interface module 210, an intent decoding module 220, a graph curation module 230, a prompt generation module 240, a language model 250, a governance module 260, a response validation module 270, a longitudinal profiling module 280, and a database 290.

The user interface module 210 is configured to present interaction interfaces and receive user inputs. The user interface module 210 renders graphical user interfaces on client devices, including text input windows, voice input controls, and multimodal input mechanisms. The user interface module 210 captures user queries, contextual signals, biometric data streams from connected sensors, or some combination thereof. The user interface module 210 transmits the captured inputs to downstream processing modules for intent decoding and reasoning. The user interface module 210 also presents reasoning outputs, explanation traces, and uncertainty indicators to the user through the rendered interface.

In one or more embodiments, the user interface module 210 generates an output report with the response and a reasoning trace. The output report includes audit logging metadata that records the reasoning process, including model version identifiers, policy version identifiers, applied constraint rule identifiers, conflict metrics, uncertainty metrics, or some combination thereof. The system applies hashing or encryption techniques to protect user privacy, wherein the output packet may be cryptographically hashed to ensure integrity and enable deterministic reconstruction, and wherein sensitive user data within the longitudinal profile may be encrypted prior to storage. The audit logging enables regulatory inspection, compliance verification, and reproducibility, while the hashing and encryption mechanisms ensure data integrity and privacy protection throughout the reasoning workflow.

The intent decoding module 220 is configured to transform inputs into normalized intent representations. The module receives raw user inputs, biometric signals, and contextual data from the user interface module. The module performs parallel feature extraction across multiple signal streams to generate feature vectors characterizing user intent. The module applies a cognitive state estimation engine to infer user objectives, domain context, and safety categories. The module outputs a structured intent representation comprising domain classification, extracted entities, inferred objectives, safety category tags, and requested output structure.

The graph curation module 230 is configured to curate one or more knowledge graphs. The module can obtain or access primary source materials, e.g., from the third-party system 130. In one or more embodiments, the graph curation module 230 can generate and update the Hierarchical Universal Knowledge Graph by ingesting primary source materials and creating or refreshing source nodes (L0) with provenance metadata, then extracting structured claim nodes (L1), normalizing concept nodes (L2), updating framework mappings (L3), revising governance overlays (L4), or some combination thereof.

Upon retrieving new or revised primary sources (e.g., enterprise repositories or external data feeds), the module adds or updates typed nodes (concept, claim, rule, framework, source, context) with timestamps, confidence weights, jurisdiction tags, and trust indicators, and then recomputes derivative nodes and their attributes. This may entail re-extracting propositions, re-normalizing concepts, and remapping framework associations to reflect the new evidence base. The module further generates new typed edges (supports, contradicts, refines, governed_by, disallowed_by, derived_from), recalculates edge weights using updated confidence and time-aware decay parameters, and reweights relationships to incorporate changes in retrieval coverage and source reliability; where applicable, it triggers downstream updates such as conflict and uncertainty recomputation and writes version-controlled audit records to preserve deterministic reproducibility of the curated graph state.

The graph curation module 230 is configured to retrieve structured subgraphs from the knowledge graph. The module receives the normalized intent representation from the intent decoding module. The module queries the hierarchical universal knowledge graph using typed relation traversal, embedding similarity search, hybrid ranking mechanisms, or some combination thereof. The module applies policy-filtered retrieval constraints to ensure compliance with governance boundaries. The module produces a structured retrieval packet containing subgraph references, coverage metrics, provenance identifiers, or some combination thereof.

The prompt generation module 240 is configured to generate candidate prompts from intent and one or more subgraphs. The prompt generation module 240 receives the intent representation and retrieved knowledge subgraphs from upstream modules. The prompt generation module 240 constructs a plurality of candidate prompts by conditioning on the structured knowledge and user intent. The prompt generation module 240 may incorporate personalization profile constraints and active framework specifications into prompt construction. The prompt generation module 240 outputs the candidate prompts for execution by the language model.

The language model 250 is configured to execute prompts and generate candidate reasoning outputs. The language model 250 inputs candidate prompts from the prompt generation module 240. The language model 250 applies one or more statistical inference models to generate candidate responses based on the prompts. The language model 250 may generate multiple candidate outputs per prompt to enable downstream validation and selection. The module outputs the candidate reasoning outputs along with associated reasoning traces for evaluation.

The governance module 260 enforces executable condition-action rules within the reasoning pipeline to ensure safety, policy compliance, and domain-of-scope adherence. It receives candidate responses and associated reasoning traces from the language model 250 and evaluates them against governance policies and rule nodes (e.g., hard and soft constraints) mapped in the Hierarchical Universal Knowledge Graph (HUKG) and active policy configurations. Hard constraints deterministically block, repair, redirect, or escalate disallowed outputs, while soft constraints penalize or repair non-compliant content within the reasoning process prior to final output selection, ensuring governance is embedded rather than applied post hoc.

Operating in parallel with the response validation module 270, the governance module independently applies executable rules and emits structured compliance indicators (e.g., which hard constraints were satisfied, which soft constraints were triggered, and what enforcement actions were taken). These indicators feed into output selection alongside framework scores, conflict metrics, and uncertainty measures, with compliance serving as a mandatory prerequisite. The module supports policy-filtered retrieval boundaries, jurisdictional overlays, and escalation triggers when conflict or uncertainty exceed predefined thresholds. It contributes to audit-grade traceability by providing rule identifiers, policy version identifiers, and enforcement actions that are included in the structured output packet and audit records, enabling deterministic reconstruction across centralized or distributed deployments while preserving governance supremacy over personalization preferences. The response validation module 270 is configured to evaluate candidates under multiple reasoning frameworks. The module receives candidate outputs and reasoning traces from the language model. The module executes a plurality of independent framework evaluators, each producing quantitative scores, structured rationales, constraint signals, and contradiction indicators. The module computes conflict metrics quantifying divergence among framework outputs. The module generates structured evaluation artifacts for use in output selection and explanation generation.

The longitudinal profiling module 280 is configured to maintain user profiles under consent and retention controls. The module receives user inputs, output hashes, and session signals from other system components. The module updates longitudinal reasoning profiles only when explicit user permission has been granted. The module encrypts profile data and enforces user-defined retention and deletion policies. The module provides profile data to other modules to enable personalization while preserving governance supremacy.

The database 290 is configured to store the hierarchical universal knowledge graph and metadata. The database maintains typed nodes and edges with associated provenance metadata, confidence weights, and framework mappings. The database supports versioned storage of knowledge elements, governance rules, and policy configurations. The database enables efficient retrieval of structured subgraphs based on intent representations and policy constraints. The database provides persistent storage for audit records, reasoning artifacts, and longitudinal user profiles.

### Hierarchical Universal Knowledge Graph

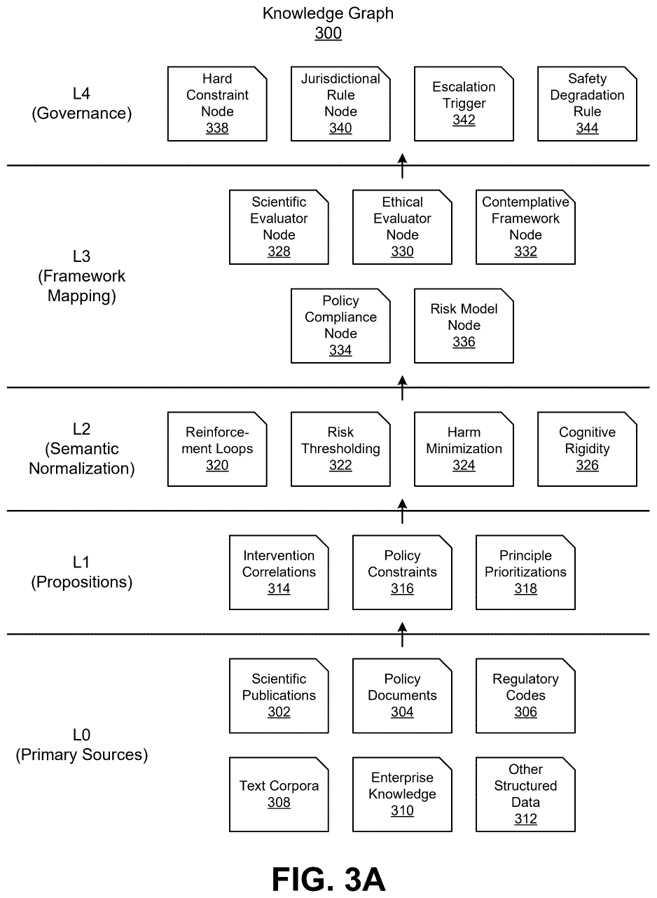

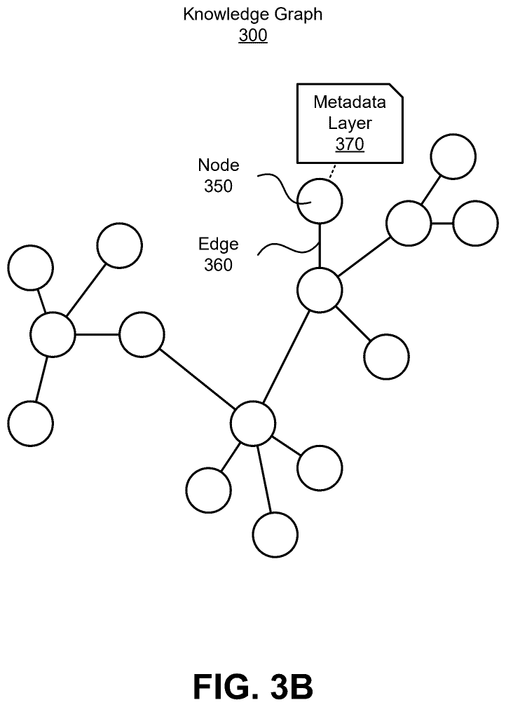

FIG. 3A illustrates the hierarchical layered structure of a knowledge graph 300 comprising primary sources at layer L0, propositions at layer L1, semantic normalization at layer L2, framework mapping at layer L3, and governance at layer L4. The knowledge graph 300 may also be referred to as a hierarchical universal knowledge graph, which synthesizes primary source materials into a hierarchical data structure with propositions, semantics, mappings, governance, or some combination thereof derived from the primary source materials.

The knowledge graph 300 comprises a plurality of typed node categories, wherein node types are not merely semantic labels but functionally distinct entities within the reasoning pipeline.

Source nodes represent external or internal source data objects from which propositions are derived. Each source node anchors knowledge elements to their originating materials, enabling traceable provenance throughout the reasoning pipeline. Source nodes may include a source identifier that uniquely references the originating document or data stream. Source nodes may further include origin metadata describing the source type, authorship, and publication context. Source nodes may also include a timestamp indicating when the source was accessed or ingested, a trust classification score reflecting the reliability of the source, and an extraction method identifier specifying the technique used to derive structured knowledge from the source.

Example source nodes include scientific publications 302, policy documents 304, regulatory codes 306, text corpora 308, enterprise knowledge 310, or other structured data 312. Scientific publications 302 represent peer-reviewed articles, research papers, or academic journals that provide empirical evidence or theoretical frameworks. Policy documents 304 represent organizational guidelines, institutional policies, or procedural manuals that define operational standards or decision-making frameworks. Regulatory codes 306 represent legal statutes, administrative regulations, or compliance requirements that establish mandatory constraints or permissible actions. Text corpora 308 represent large collections of written or spoken language samples that provide linguistic patterns or domain-specific terminology. Enterprise knowledge 310 represents proprietary information, internal documentation, or organizational expertise that reflects institutional practices or business intelligence. Other structured data 312 represents databases, ontologies, or formatted datasets that provide factual information or relational mappings.

Concept nodes represent normalized abstractions of ideas, entities, states, or phenomena within the knowledge graph. These nodes serve as canonical representations that unify related mentions across multiple sources and contexts. Concept nodes may include a canonical label providing a standardized name for the concept. Concept nodes may further include a semantic embedding vector that encodes the concept's meaning in a high-dimensional space for similarity computation. Concept nodes may also include a confidence weight reflecting the certainty of the concept's definition, a version identifier tracking changes to the concept over time, and a linked abstraction hierarchy connecting the concept to broader or narrower related concepts.

Concept nodes include, for example, intervention correlations 314, policy constraints 316, principle prioritizations 318, reinforcement loops 320, risk thresholding 322, harm minimization 324, and cognitive rigidity 326. Intervention correlations 314 represent causal or statistical relationships between actions and outcomes that inform adaptive behavioral guidance. Policy constraints 316 represent organizational or regulatory boundaries that limit permissible reasoning pathways or output content. Principle prioritizations 318 represent ethical or normative hierarchies that determine relative weighting of competing values in decision-making. Reinforcement loops 320 represent recurrent behavioral or cognitive patterns that amplify or perpetuate specific states through feedback mechanisms. Risk thresholding 322 represent quantitative or qualitative boundaries that trigger escalation, intervention, or constraint activation based on assessed harm likelihood. Harm minimization 324 represent strategies or constraints designed to reduce adverse impacts on affected agents or stakeholders. Cognitive rigidity 326 represents structural inflexibility in reasoning patterns characterized by low hypothesis diversity or circular premise reinforcement.

Framework nodes represent formal reasoning frameworks executable within the multi-framework evaluator subsystem. These nodes define the criteria and logic by which candidate outputs are assessed under different evaluative lenses. Framework nodes may encode evaluation criteria specifying the standards against which outputs are judged. Framework nodes may further encode scoring functions that compute quantitative assessments of candidate outputs, and constraint hooks that link the framework to applicable governance rules. Framework nodes may also encode priority weighting parameters that determine the relative influence of the framework in output selection, and jurisdictional applicability metadata specifying the legal or regulatory contexts in which the framework applies.

Framework nodes may include, for example, a scientific evaluator node 328, an ethical evaluator node 330, a contemplative framework node 332, a policy compliance node 334, and a risk model node 336. A scientific evaluator node 328 represents a framework that assesses candidate outputs for empirical plausibility, consistency with established scientific principles, or alignment with evidence-based reasoning standards. An ethical evaluator node 330 represents a framework that evaluates candidate outputs for moral acceptability, fairness, stakeholder impact, or alignment with normative ethical principles. A contemplative framework node 332 represents a framework that models cognitive-state transitions, behavioral optimization pathways, or adaptive guidance strategies operationalized as executable reasoning logic. A policy compliance node 334 represents a framework that verifies candidate outputs against organizational policies, institutional guidelines, or procedural requirements. A risk model node 336 represents a framework that quantifies potential harms, safety violations, or adverse outcomes associated with candidate outputs.

Rule nodes represent executable governance constraints expressed as structured condition-action logic. These nodes define the enforceable boundaries within which the reasoning system must operate. Rule nodes may include trigger conditions that specify the circumstances under which the rule activates. Rule nodes may further include an enforcement action defining the system's response when the rule is triggered, such as blocking, repairing, redirecting, or escalating a candidate output. Rule nodes may also include a priority level determining the order in which rules are evaluated, an applicable scope specifying the domains or contexts to which the rule applies, and version control metadata enabling tracking of rule changes over time.

Rule nodes may include, for example, a hard constraint node 338, a jurisdictional rule node 340, an escalation trigger 342, and a safety degradation rule 344. A hard constraint node 338 represents a mandatory prohibition that blocks, refuses, or redirects outputs violating safety boundaries, regulatory requirements, or domain-of-scope limitations. A jurisdictional rule node 340 represents a geographically or legally scoped constraint that enforces region-specific regulations, compliance standards, or policy overlays applicable within particular legal frameworks. An escalation trigger 342 represents a conditional threshold that activates elevated safety behavior, human review, or conservative output narrowing when conflict scores, uncertainty metrics, or sensitive intent classifications exceed predefined limits. A safety degradation rule 344 represents a dynamic constraint that reduces output assertiveness, increases clarification prompts, or narrows recommendation scope when system confidence falls below acceptable levels or when potential harm likelihood rises above safety thresholds.

Context nodes represent situational or environmental parameters affecting reasoning evaluation. These nodes capture the specific circumstances under which reasoning occurs, enabling context-sensitive application of knowledge and constraints. Context nodes may encode user domain context describing the subject matter area relevant to the current query. Context nodes may further encode jurisdiction information specifying the legal or regulatory framework applicable to the reasoning task, and policy regime identifiers indicating which governance policies are active. Context nodes may also encode temporal context marking the time at which reasoning occurs, and operational constraints defining resource limits or performance requirements for the reasoning process.

FIG. 3B illustrates the knowledge graph structure comprising nodes, edges, and a metadata layer. The figure depicts a representative portion of the knowledge graph showing the interconnected nature of typed nodes and the relationships encoded through typed edges. The metadata layer overlays the graph structure to provide additional context, versioning information, and provenance tracking for each element. This visualization demonstrates how the knowledge graph integrates structural relationships with rich metadata to enable reasoning-aware operations.

Edges in the HUKG are explicitly typed and may carry directional, weighted, and temporal properties. Each edge type encodes a specific semantic relationship between connected nodes, enabling the system to traverse the graph according to logical inference rules. Directional properties specify whether relationships are unidirectional or bidirectional, affecting how reasoning propagates through the graph. Weighted properties assign numerical values reflecting the strength or confidence of each relationship. Temporal properties track when relationships were established or last validated, supporting time-aware reasoning and decay modeling.

Edge categories may include supports, contradicts, refines, governed_by, requires, disallowed_by, and derived_from, wherein each edge may include edge weight, temporal scope, confidence adjustment factor, and jurisdictional applicability. The supports edge indicates that one node provides evidential or logical support for another node. The contradicts edge encodes logical opposition or incompatibility between nodes. The refines edge expresses specialization or abstraction hierarchy relationships. The governed_by edge links concepts or claims to applicable constraint rules. The requires edge encodes dependency relationships where one node necessitates another. The disallowed_by edge maps content to blocking constraints. The derived_from edge links claims or concepts to their provenance sources.

This typed relational structure enables contradiction detection, conflict scoring, and rule enforcement at graph level. The system traverses typed edges to identify contradictory claims within retrieved subgraphs, computing contradiction density metrics that feed into conflict scores. The governance edges enable direct enforcement of constraints by linking knowledge elements to applicable rules. The provenance edges support audit reconstruction by tracing reasoning outputs back to their source materials. The typed structure allows the system to perform graph-level operations that would be impossible with untyped or weakly-typed knowledge representations.

Each node within the HUKG may include globally unique identifier, timestamp of creation or last update, confidence weight, provenance identifier linking to source node, trust classification score, optional jurisdiction tag, version identifier, and decay or aging parameter. The globally unique identifier enables unambiguous reference to each node across distributed system components. The timestamp tracks when the node was created or most recently modified, supporting temporal reasoning and version control. The confidence weight quantifies the certainty associated with the node's content, derived from source reliability and extraction quality. The provenance identifier links the node to its originating source, enabling traceable reasoning. The trust classification score reflects the reliability of the source from which the node was derived.

The inclusion of confidence and provenance metadata enables uncertainty propagation and audit reconstruction. Confidence weights propagate through reasoning chains, allowing the system to compute aggregate uncertainty for candidate outputs based on the confidence of underlying knowledge elements. Provenance identifiers enable the system to generate explanation traces that link output claims back to specific source materials. The metadata supports deterministic reconstruction of reasoning processes by preserving the complete context in which each knowledge element was used. This metadata-rich structure distinguishes the HUKG from conventional knowledge graphs that lack the metadata necessary for uncertainty-aware reasoning and compliance-grade auditability.

The HUKG is hierarchically layered to enable structured reasoning abstraction, wherein L0 stores references to unprocessed or minimally processed source artifacts, L1 contains structured claim nodes derived from L0 via extraction pipelines, L2 represents abstracted and canonicalized conceptual entities, L3 maps concepts and claims to executable reasoning frameworks, and L4 contains rule nodes and constraint mappings applied during evaluation. This layered architecture separates raw source materials from progressively refined knowledge representations, enabling the system to operate at the appropriate level of abstraction for each reasoning task. The L0 layer preserves original source materials for audit and verification purposes. The L1 layer extracts structured propositions from sources through natural language processing and information extraction techniques. The L2 layer normalizes and canonicalizes concepts to create unified representations across multiple sources. The L3 layer associates knowledge elements with applicable reasoning frameworks. The L4 layer overlays governance constraints that bound permissible reasoning operations.

Unlike conventional knowledge graphs that serve retrieval purposes only, the HUKG is directly integrated into multi-framework evaluation, conflict detection computation, constraint execution, uncertainty propagation, and explanation trace generation. The graph structure is not merely a passive data store but an active component of the reasoning pipeline. Framework evaluators query the graph to retrieve relevant knowledge elements and assess their consistency with evaluative criteria. The conflict detection module traverses contradiction edges to identify inconsistencies within retrieved subgraphs. The constraint execution engine follows governance edges to enforce applicable rules. The uncertainty propagation module aggregates confidence weights from referenced nodes. The explanation trace generator follows provenance edges to construct machine-readable reasoning artifacts.

The graph structure enables contradiction density measurement, dependency traversal, policy-scoped retrieval, confidence-weighted reasoning, and dynamic decay modeling. Contradiction density is computed by analyzing the proportion of contradicts edges within a retrieved subgraph relative to the total number of edges. Dependency traversal follows requires edges to ensure that all prerequisite knowledge elements are included in reasoning. Policy-scoped retrieval filters graph queries based on jurisdictional tags and governance constraints to ensure compliance. Confidence-weighted reasoning adjusts the influence of each knowledge element according to its confidence weight. Dynamic decay modeling applies time-dependent attenuation to confidence weights based on decay parameters, reducing the influence of stale knowledge over time.

The HUKG differs from conventional graph databases and semantic knowledge graphs in that it encodes executable governance rules alongside knowledge, supports cross-framework evaluation mapping, incorporates confidence and decay modeling, enables formal contradiction scoring, and is integrated into candidate validation rather than passive retrieval. Conventional knowledge graphs typically store only factual assertions and semantic relationships without governance constraints. The HUKG embeds rule nodes and governance edges that enable enforceable constraint execution. Conventional graphs lack the framework mapping layer that enables multi-perspective evaluation. The HUKG's confidence and decay parameters enable uncertainty-aware reasoning that adapts to knowledge staleness. The formal contradiction scoring enabled by typed edges provides quantitative measures of knowledge consistency. The integration into candidate validation transforms the graph from a retrieval index into a reasoning substrate.

The HUKG therefore functions as a reasoning-aware, constraint-integrated knowledge substrate rather than a static semantic index. The graph actively participates in reasoning operations through its typed structure, rich metadata, hierarchical layering, and integration with framework evaluators and governance engines. This architecture enables the system to perform structured reasoning under executable constraints with audit-grade traceability, distinguishing it from conventional knowledge graph applications that support only retrieval and simple inference.

### Constraint-Governed Neuro-Symbolic Reasoning Infrastructure

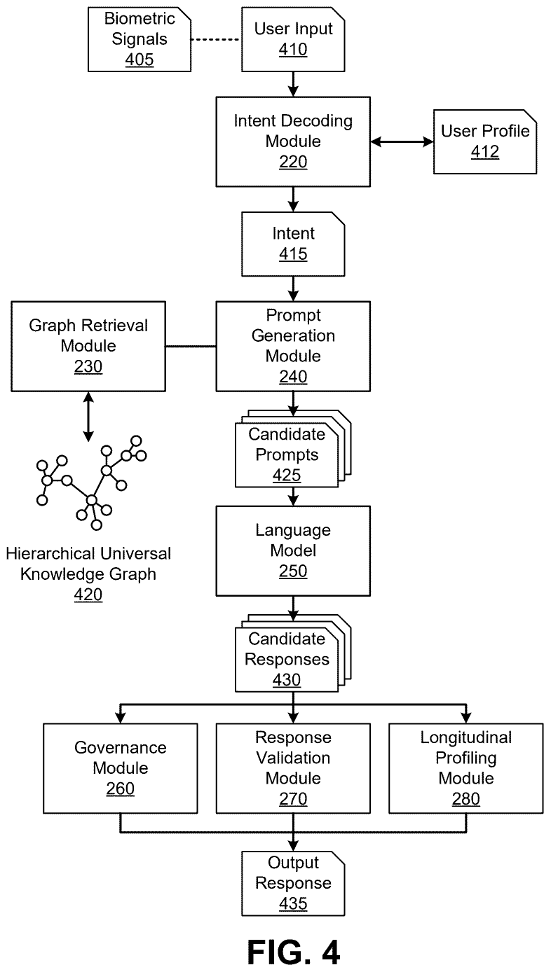

FIG. 4 illustrates an illustrative workflow of the reasoning infrastructure deployed by the analytics system 110, according to some embodiments. The workflow may include intent decoding, graph retrieval, prompt generation, language model execution, and parallel processing by governance, response validation, and longitudinal profiling modules to produce an output response.

The system retrieves one or more structured subgraphs from the Hierarchical Universal Knowledge Graph (HUKG) 420, wherein the HUKG comprises typed nodes and edges storing provenance metadata, confidence weights, and framework mappings. The retrieval process queries the graph using the normalized intent representation 415 to identify knowledge elements relevant to the user's query. The graph retrieval module 230 applies typed relation traversal to follow semantic relationships between nodes, embedding similarity search to identify conceptually related elements, and hybrid ranking mechanisms to prioritize the most relevant subgraphs. Policy-filtered retrieval constraints ensure that only knowledge elements consistent with applicable governance rules are included in the retrieved subgraphs. The retrieved subgraphs include provenance metadata linking each element to its source, confidence weights quantifying the reliability of each knowledge element, and framework mappings indicating which reasoning frameworks are applicable to each element.

The graph retrieval module 230 accesses the HUKG 420 and retrieves relevant subgraphs based on the intent representation 415. The module receives the normalized intent representation from the intent decoding module 220, which specifies the domain classification, extracted entities, inferred objectives, safety category tags, and requested output structure. The graph retrieval module constructs a query that combines the intent representation with user profile 412 preferences to identify the most relevant portions of the knowledge graph. The module produces a structured retrieval packet containing subgraph references, coverage metrics indicating the completeness of the retrieved knowledge, and provenance identifiers linking each retrieved element to its originating source. This retrieval packet is transmitted to the prompt generation module 240 for use in constructing candidate prompts.

The prompt generation module 240 generates a plurality of candidate prompts 425 based on the user intent feature vector and the hierarchical universal knowledge graph 420. Each candidate prompt is constructed by conditioning on the retrieved knowledge subgraphs and the normalized intent representation 415 to create a structured input for the language model 250. The prompt generation process incorporates personalization constraints from the user profile 412, active framework specifications, and governance boundaries to ensure that generated prompts align with user preferences and policy requirements. Multiple candidate prompts may be generated to explore different reasoning pathways, to enable downstream validation through comparison, or to increase the diversity of candidate responses available for evaluation. The candidate prompts 425 are formatted according to the input requirements of the language model and transmitted for execution.

The system generates a plurality of candidate reasoning outputs using one or more statistical inference models conditioned on the retrieved subgraphs and intent representation. The language model 250 receives the candidate prompts 425 from the prompt generation module 240 and applies statistical inference over its learned parameter space to generate natural language responses. Each execution produces a candidate response along with an associated reasoning trace that documents the intermediate reasoning steps, referenced knowledge elements from the HUKG 420, and applied inference rules. The language model may generate multiple candidate responses 430 per prompt through sampling techniques, beam search, or other generation strategies to increase the diversity of outputs available for validation. The statistical inference process conditions on both the structured knowledge from the retrieved subgraphs and the normalized intent representation to ensure that generated responses are grounded in the knowledge graph and aligned with user intent.

The language model 250 executes each candidate prompt on a machine-learning model, yielding at least one candidate response 430 per candidate prompt. The language model applies transformer-based architectures, recurrent neural networks, or other statistical inference models to generate responses conditioned on the structured prompts. Each execution produces a candidate response comprising natural language text along with an associated reasoning trace that documents the generation process. The reasoning trace includes references to knowledge graph nodes accessed during generation, intermediate reasoning steps performed by the model, and confidence scores associated with generated content. The candidate responses 430 and their associated reasoning traces are transmitted in parallel to the governance module 260, the response validation module 270, and the longitudinal profiling module 280 for evaluation and scoring.

The analytics system 110 further leverages the governance module 260, the response validation module 270, and the longitudinal profiling module 280 to score and rank the candidate responses 430, for eventual selection and output to the client device 120. The governance module 260 evaluates each candidate response against executable condition-action governance rules to enforce hard constraints that block disallowed outputs and soft constraints that penalize non-compliant candidates. The response validation module 270 executes multiple independent framework evaluators to assess each candidate response under distinct reasoning frameworks, computing conflict scores that quantify divergence among evaluator outputs and uncertainty scores that aggregate coverage metrics, variance scores, and rule violation indicators. The longitudinal profiling module 280 updates the user profile 412 based on the selected response and session signals, subject to explicit user consent and retention controls. The system ranks candidate responses according to a composite utility function combining framework evaluation scores, conflict-adjusted rankings, uncertainty weightings, governance compliance states, and longitudinal profile preferences, selecting the highest-ranked response that satisfies all governance constraints for transmission as the output response 435 to the client device 120.

### Intent Decoding

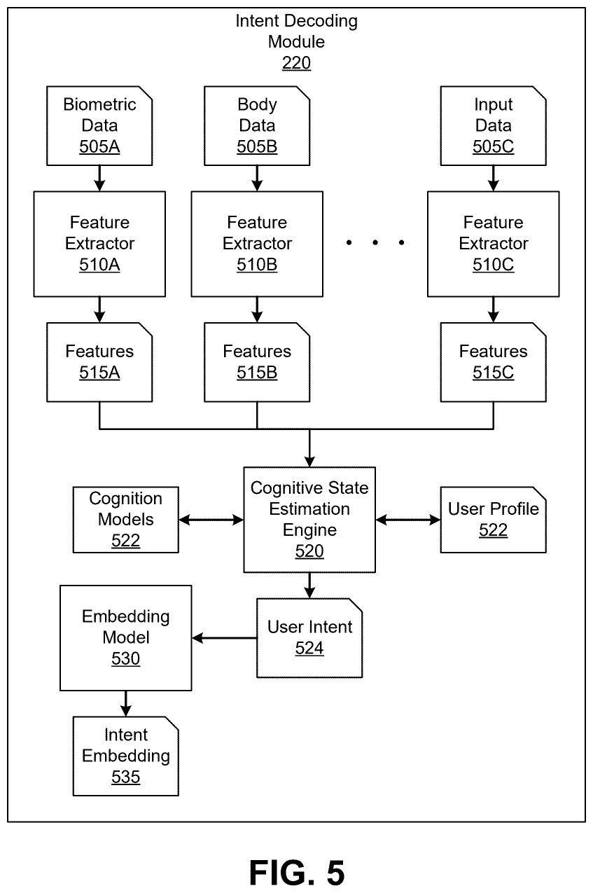

FIG. 5 illustrates a workflow of an intent decoding module 220, according to some embodiments. The intent decoding module 220 includes a plurality of feature extractors, e.g., feature extractors 510A, 510B, and 510C for biometric data 505A, body data 505B, and input data 505C, respectively. The intent decoding module 220 also includes a cognitive state estimation engine 520 and an embedding model 530 to generate an intent embedding 535.

The intent decoding module 220 receives multiple parallel input streams and processes them through dedicated feature extraction pathways to generate a unified representation of user intent. The module integrates multimodal signals to infer cognitive state, domain context, and user objectives. The resulting intent embedding 535 serves as a structured input for downstream knowledge retrieval and prompt generation operations.

The system receives an input query and associated contextual signals, including user parameters, domain constraints, and applicable governance policies. The input query may comprise natural language text, voice commands, or other forms of user interaction captured through the client device 120. Contextual signals include user-specific parameters such as historical interaction patterns, domain constraints specifying the subject matter area or scope of the query, and applicable governance policies that define safety boundaries and compliance requirements. These signals are captured in parallel and transmitted to the intent decoding module 220 for processing. The system may also receive biometric signals from health sensors 130 coupled to the user, providing physiological measurements that inform cognitive state estimation.

The intent decoding module 220 transforms the input into a normalized intent representation 415, comprising structured elements such as domain classification, extracted entities, inferred objectives, safety category tags, and requested output structure. Domain classification assigns the query to one or more subject matter categories that determine which knowledge graph regions and framework evaluators are most relevant. Extracted entities identify specific concepts, individuals, locations, or other named elements referenced in the query. Inferred objectives represent the system's interpretation of what the user seeks to accomplish, such as obtaining information, making a decision, or receiving guidance. Safety category tags flag queries that may require elevated scrutiny or constraint enforcement based on content sensitivity or potential harm. Requested output structure specifies the format and level of detail the user expects in the response.

The intent decoding module 220 accesses biometric data 505A, body data 505B, and input data 505C, and performs parallel feature extraction for each signal stream through feature extractors 510A, 510B, 510C, yielding feature vectors 515A, 515B, 515C characterizing user intent during the session.

Each feature extractor operates independently on its assigned input modality to generate modality-specific representations. The biometric data feature extractor 510A processes physiological signals such as heart rate variability, respiratory patterns, or electroencephalography measurements to extract features indicative of emotional state, attention stability, or cognitive load. The body data feature extractor 510B processes motion, posture, or interaction telemetry to extract features reflecting user engagement or behavioral patterns. The input data feature extractor 510C processes textual or voice input to extract semantic features, sentiment indicators, and linguistic patterns. The resulting feature vectors 515A, 515B, 515C are transmitted to the cognitive state estimation engine 520 for integration.

The cognitive state estimation engine 520 processes the extracted features 515A, 515B, 515C in conjunction with cognition models 522 and user profile 522 data to generate a user intent representation 524. The cognitive state estimation engine applies multimodal fusion techniques to integrate feature vectors from different input modalities into a unified representation of the user's cognitive and emotional state. The cognition models 522 comprise trained statistical models or rule-based systems that map feature patterns to cognitive state categories such as focused attention, cognitive overload, emotional arousal, or distraction. The user profile 522 provides historical context about the user's typical interaction patterns, preferences, and baseline cognitive states. The engine combines current session features with profile data and cognition model predictions to generate the user intent representation 524, which encodes the user's query, inferred goals, cognitive state, and contextual parameters.

The embedding model 530 converts the user intent representation 524 into an intent embedding 535 suitable for downstream processing. The embedding model applies dimensionality reduction, semantic encoding, or learned transformation techniques to map the structured intent representation into a dense vector space that facilitates efficient similarity computation and knowledge graph querying. The intent embedding 535 preserves the semantic content of the user's query while encoding cognitive state, domain context, and safety category information in a format compatible with the graph retrieval module 230 and prompt generation module 240. The intent embedding serves as the primary query representation for retrieving relevant knowledge subgraphs from the HUKG 420 and for conditioning candidate prompt generation.

### Response Validation

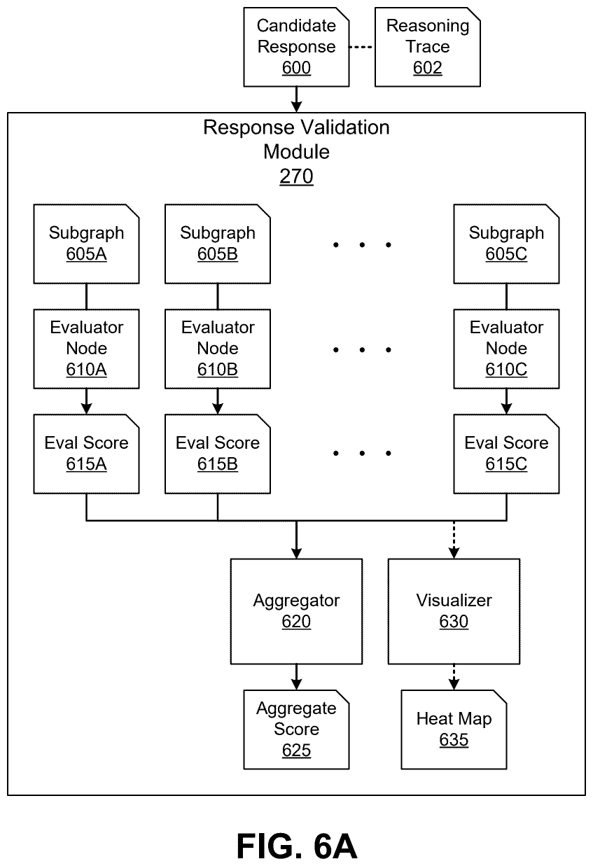

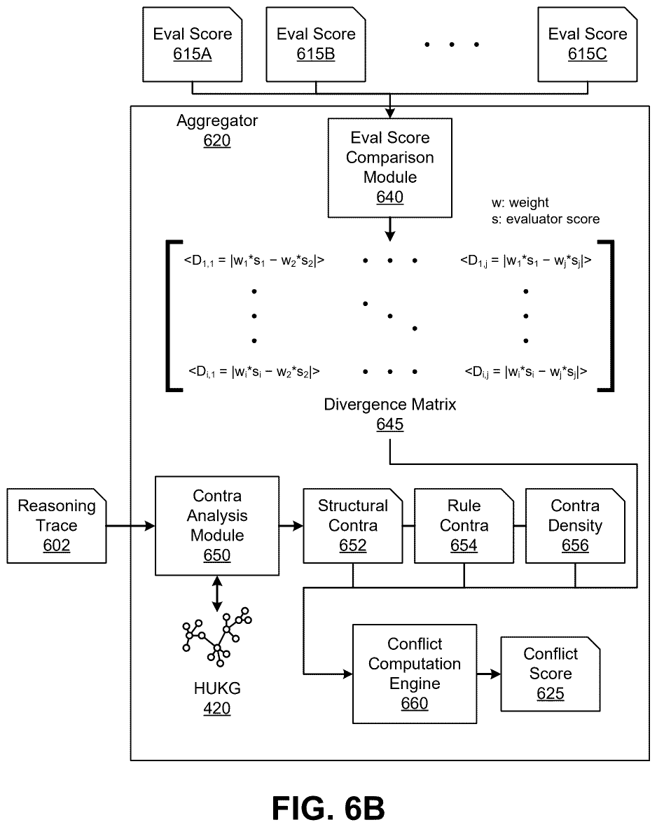

FIG. 6A illustrates a response validation module 270 that processes candidate responses 600 and reasoning traces 602 through multiple evaluator nodes to generate evaluation scores, which are aggregated and visualized as a heat map 635. The response validation module 270 receives candidate responses 600 from the language model 250 along with their associated reasoning traces 602 that document the generation process. The module routes each candidate response through multiple parallel evaluation pathways, each corresponding to a distinct reasoning framework such as scientific plausibility, ethical acceptability, policy compliance, or risk assessment. Each evaluator node 610A, 610B, 610C applies framework-specific criteria to assess the candidate response and generates an evaluation score 615A, 615B, 615C that quantifies the degree of alignment between the response and the framework's standards. The evaluation scores are collected by an aggregator 620 that computes a composite aggregate score 625 representing the overall quality of the candidate response across all frameworks. A visualizer 630 may generate a heat map 635 representation that displays the evaluation results in a graphical format, enabling visual inspection of framework-specific strengths and weaknesses for each candidate response.

The system evaluates each candidate output under a plurality of independent reasoning frameworks, wherein each framework produces structured evaluation artifacts including quantitative scores, detected contradictions, constraint signals, and explanatory rationale. Each framework evaluator operates independently without knowledge of other evaluators' assessments, ensuring that framework-specific criteria are applied consistently and without bias from other perspectives. The quantitative scores provide numerical assessments of candidate quality according to each framework's standards, typically normalized to a common scale to enable cross-framework comparison. Detected contradictions identify inconsistencies between the candidate response and knowledge elements in the retrieved subgraph, flagging potential factual errors or logical incoherence. Constraint signals indicate whether the candidate response violates any governance rules or policy boundaries associated with the framework. Explanatory rationale provides structured justification for the evaluation scores, documenting which aspects of the candidate response contributed positively or negatively to the assessment.

The response validation module 270 processes candidate responses 600 and reasoning traces 602 through multiple subgraphs 605A, 605B, 605C, each associated with an evaluator node 610A, 610B, 610C that generates an evaluation score 615A, 615B, 615C. Each subgraph 605A, 605B, 605C represents a portion of the hierarchical universal knowledge graph 420 that is relevant to a particular reasoning framework, containing framework-specific knowledge elements, evaluation criteria, and constraint mappings. The evaluator nodes 610A, 610B, 610C access their respective subgraphs to retrieve framework-specific knowledge that informs the evaluation process, comparing the candidate response against established standards, empirical evidence, or normative principles encoded in the subgraph. Each evaluator node generates an evaluation score 615A, 615B, 615C that reflects the degree of consistency between the candidate response and the framework-specific knowledge, with higher scores indicating stronger alignment and lower scores indicating potential conflicts or deficiencies. The evaluation scores are transmitted to the aggregator 620 for combination into a composite assessment.

An aggregator 620 collects the evaluation scores 615A, 615B, 615C and generates an aggregate score 625, while a visualizer 630 may generate a heat map 635 representation of the evaluation results. The aggregator 620 applies a weighted combination function to the individual framework scores, where weights may be determined by user preferences in the longitudinal profile, framework priority settings, or domain-specific importance factors. The aggregate score 625 provides a single numerical value representing the overall quality of the candidate response across all evaluated frameworks, enabling straightforward comparison and ranking of multiple candidate responses. The visualizer 630 generates a heat map 635 that displays evaluation scores in a color-coded matrix format, where rows represent different candidate responses, columns represent different framework evaluators, and color intensity indicates score magnitude. The heat map 635 enables rapid visual identification of candidate responses that perform well across all frameworks versus those that exhibit strong performance in some frameworks but weak performance in others.

The system applies executable constraint logic via a governance engine, wherein hard constraints block or redirect disallowed outputs and soft constraints repair or penalize non-compliant candidates within the reasoning process itself. The governance engine evaluates each candidate response against a set of executable condition-action rules that encode safety boundaries, regulatory requirements, domain-of-scope limitations, and policy compliance standards. Hard constraints represent mandatory prohibitions that cannot be overridden, triggering automatic blocking of candidate responses that violate critical safety or legal requirements, redirection to safer alternative responses, or escalation to human review when appropriate. Soft constraints represent preferences or optimization criteria that influence candidate ranking without absolute prohibition, applying weighted penalties to candidate responses that exhibit undesirable characteristics or performing automated repairs that modify response content to improve compliance while preserving semantic intent. The constraint execution occurs within the reasoning pipeline prior to final output selection, ensuring that governance enforcement is deterministic and architecturally embedded rather than applied as a post-hoc filter.

A governance module 260 operates in parallel with the response validation module 270 to enforce governance rules on candidate responses 430. The governance module 260 receives the same candidate responses 430 that are transmitted to the response validation module 270, enabling simultaneous evaluation of framework alignment and governance compliance. The governance module applies its executable constraint logic independently of the framework evaluators, ensuring that governance enforcement is not influenced by framework-specific assessments and that mandatory safety boundaries are enforced regardless of framework scores. The governance module generates compliance indicators that specify whether each candidate response satisfies all hard constraints, which soft constraints were triggered, and what enforcement actions were taken. These compliance indicators are combined with framework evaluation scores and conflict metrics during the final output selection process, with governance compliance serving as a mandatory prerequisite for candidate selection.

FIG. 6B illustrates an aggregator 620 that computes a divergence matrix 645 from evaluation scores 615A, 615B, 615C, feeds the matrix to a contra analysis module 650 that generates structural contra 652, rule contra 654, and contra density 656 metrics, which are processed by a conflict computation engine 660 to produce a conflict score 625. The aggregator 620 receives evaluation scores from multiple framework evaluators and computes pairwise differences between scores to quantify the degree of disagreement among evaluators. The divergence matrix 645 is a square matrix where each element represents the absolute difference between scores from two different evaluators, providing a comprehensive view of cross-framework disagreement patterns. The contra analysis module 650 processes the divergence matrix 645 in conjunction with reasoning traces 602 and the HUKG 420 to identify the sources and nature of evaluator disagreement, distinguishing between structural contradictions arising from inconsistent knowledge elements, rule contradictions arising from conflicting governance constraints, and general disagreement arising from different evaluative priorities. The conflict computation engine 660 aggregates the structural contra 652, rule contra 654, and contra density 656 metrics into a single conflict score 625 that quantifies the overall degree of cross-framework disagreement for each candidate response.

The system detects and quantifies cross-framework conflict, including divergence between evaluators, contradiction density within the knowledge subgraph, and rule-collision events. Divergence between evaluators is measured by computing pairwise score differences and identifying cases where evaluators produce substantially different assessments of the same candidate response, indicating fundamental disagreement about response quality or appropriateness. Contradiction density within the knowledge subgraph is computed by analyzing the retrieved subgraph for contradicts edges connecting knowledge elements referenced in the candidate response, with higher contradiction density indicating that the response relies on mutually inconsistent knowledge claims. Rule-collision events occur when different governance rules or framework-specific constraints produce conflicting recommendations about whether a candidate response should be permitted, repaired, or blocked. The system quantifies these conflict sources through structured metrics that enable explicit detection of epistemic instability and inform downstream decisions about output selection, branching, or escalation.

An eval score comparison module 640 computes pairwise divergence between evaluation scores 615A, 615B, 615C to generate a divergence matrix 645. The eval score comparison module 640 receives evaluation scores from all active framework evaluators and systematically compares each pair of scores to quantify the degree of disagreement. For each pair of evaluators i and j, the module computes a divergence value D_ij equal to the weighted absolute difference between their scores, where weights may reflect the relative importance or reliability of each evaluator. The divergence matrix 645 is populated with these pairwise divergence values, creating a symmetric matrix that captures the complete pattern of cross-framework disagreement. The divergence matrix 645 is transmitted to the contra analysis module 650 for further processing to identify the structural and logical sources of evaluator disagreement.

A contra analysis module 650 processes the divergence matrix 645 in conjunction with reasoning traces 602 and the HUKG 420 to generate structural contra 652, rule contra 654, and contra density 656 metrics. The contra analysis module 650 examines the reasoning traces 602 to identify which knowledge elements from the HUKG 420 were referenced by each framework evaluator during assessment, enabling attribution of evaluator disagreement to specific knowledge claims or inference steps. Structural contra 652 metrics quantify contradictions arising from inconsistent knowledge elements within the retrieved subgraph, computed by counting contradicts edges between referenced nodes and weighting by the strength of the contradiction relationship. Rule contra 654 metrics quantify conflicts arising from incompatible governance rules or framework-specific constraints, computed by detecting cases where different rules produce opposing enforcement recommendations for the same candidate response. Contra density 656 metrics aggregate structural and rule contradictions into a normalized measure of overall inconsistency within the reasoning context, providing a single value that characterizes the degree of epistemic instability affecting the candidate response evaluation.

A conflict computation engine 660 aggregates these metrics to produce a conflict score 625 that quantifies the degree of disagreement among framework evaluators. The conflict computation engine 660 applies a weighted combination function to the structural contra 652, rule contra 654, and contra density 656 metrics, where weights are configured based on the relative importance of each conflict source in determining overall epistemic reliability. The conflict score 625 is computed as a normalized value typically ranging from zero to one, where zero indicates perfect agreement among all evaluators and one indicates maximal disagreement or contradiction. The conflict score 625 is transmitted to the uncertainty propagation subsystem where it contributes to the overall uncertainty representation, and to the output selection module where it influences candidate ranking by penalizing responses that exhibit high cross-framework disagreement.

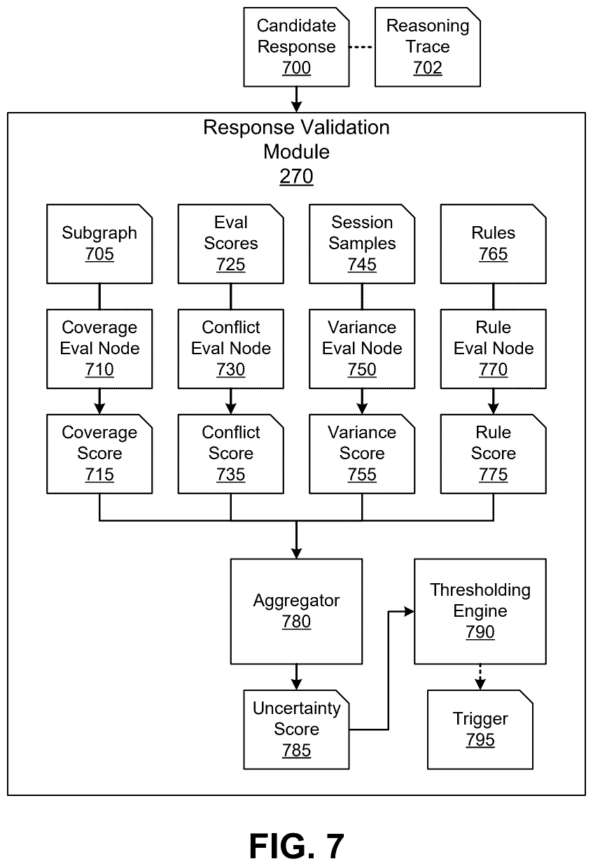

FIG. 7 illustrates a response validation module 270 that computes coverage scores 715, conflict scores 735, variance scores 755, and rule scores 775 from subgraphs 705, evaluation scores 725, session samples 745, and rules 765, aggregates these scores to generate an uncertainty score 785, and applies thresholding to generate triggers 795. The response validation module 270 operates multiple parallel evaluation pathways that each assess a different dimension of candidate response reliability and epistemic stability. A coverage eval node 710 analyzes the retrieved subgraph 705 to compute a coverage score 715 that quantifies the completeness of knowledge available to support the candidate response. A conflict eval node 730 processes evaluation scores 725 from multiple framework evaluators to compute a conflict score 735 that quantifies cross-framework disagreement. A variance eval node 750 analyzes session samples 745 representing multiple candidate responses or generation attempts to compute a variance score 755 that quantifies the stability of the generation process. A rule eval node 770 evaluates the candidate response against governance rules 765 to compute a rule score 775 that quantifies the degree of constraint violations or compliance issues. An aggregator 780 combines these component scores into a unified uncertainty score 785, and a thresholding engine 790 compares the uncertainty score against predefined thresholds to generate triggers 795 for safety-mode degradation or escalation.

The system computes and propagates structured uncertainty metrics, derived from retrieval coverage, evaluator disagreement, constraint violations, and model variance signals. Retrieval coverage metrics quantify the completeness of knowledge available in the retrieved subgraph relative to the information required to fully address the user's query, with lower coverage indicating higher epistemic uncertainty due to missing or incomplete knowledge. Evaluator disagreement metrics quantify the degree of cross-framework conflict as measured by divergence in evaluation scores, with higher disagreement indicating uncertainty about which evaluative perspective should be prioritized. Constraint violation metrics quantify the severity and frequency of governance rule violations detected in the candidate response, with more violations indicating higher uncertainty about whether the response can be safely presented. Model variance signals quantify the stability of the generation process by measuring the diversity or dispersion of candidate responses generated from similar prompts, with higher variance indicating lower confidence in any single response. These component uncertainty metrics are propagated through the reasoning pipeline and combined into a structured uncertainty representation that influences output selection, explanation generation, and safety-mode behavior.

A coverage eval node 710 computes a coverage score 715 based on the retrieved subgraph 705, a conflict eval node 730 computes a conflict score 735 based on evaluation scores 725, a variance eval node 750 computes a variance score 755 based on session samples 745, and a rule eval node 770 computes a rule score 775 based on applied rules 765. The coverage eval node 710 analyzes the retrieved subgraph 705 to identify gaps or missing knowledge elements that would be required to fully support the candidate response, computing the coverage score 715 as the ratio of available knowledge to required knowledge. The conflict eval node 730 receives evaluation scores 725 from multiple framework evaluators and computes the conflict score 735 by measuring pairwise score divergence and aggregating across all evaluator pairs. The variance eval node 750 analyzes session samples 745 representing multiple candidate responses or generation attempts to compute the variance score 755 as a measure of generation stability, with higher variance indicating lower confidence. The rule eval node 770 evaluates the candidate response against governance rules 765 to compute the rule score 775 as a weighted sum of constraint violations, with higher scores indicating more severe or numerous violations.

An aggregator 780 combines the coverage score 715, conflict score 735, variance score 755, and rule score 775 to generate an uncertainty score 785. The aggregator 780 applies a weighted combination function where each component score is multiplied by a configurable weight parameter that reflects the relative importance of that uncertainty source in the current reasoning context. The weights may be adjusted based on domain-specific requirements, user preferences, or deployment environment characteristics to emphasize particular uncertainty dimensions. The uncertainty score 785 is computed as a normalized value typically ranging from zero to one, where zero indicates maximum confidence and one indicates maximum uncertainty. The uncertainty score 785 is transmitted to downstream modules where it influences output selection by penalizing high-uncertainty candidates, modulates explanation generation by triggering more detailed justifications for uncertain responses, and controls safety-mode behavior by activating conservative fallback strategies when uncertainty exceeds acceptable thresholds.

A thresholding engine 790 compares the uncertainty score 785 against predefined thresholds to generate triggers 795 for safety-mode degradation or escalation. The thresholding engine 790 maintains multiple threshold values corresponding to different levels of uncertainty tolerance, with lower thresholds triggering mild interventions such as increased explanation detail and higher thresholds triggering more aggressive interventions such as output blocking or human escalation. When the uncertainty score 785 exceeds a first threshold, the system may generate a trigger 795 that activates enhanced explanation generation, providing more detailed justification and uncertainty disclosure in the output packet. When the uncertainty score exceeds a second, higher threshold, the system may generate a trigger 795 that activates safety-mode degradation, reducing output assertiveness, narrowing recommendation scope, or requesting additional user clarification. When the uncertainty score exceeds a third, critical threshold, the system may generate a trigger 795 that blocks output generation entirely and escalates to human review or provides a conservative fallback response. The triggers 795 are transmitted to the output selection module and user interface module to control downstream behavior.

The system selects a validated output based on combined framework scores, quantified conflict measures, propagated uncertainty values, and policy priorities. The output selection process applies a composite utility function that integrates evaluation scores from all framework evaluators, conflict scores quantifying cross-framework disagreement, uncertainty scores aggregating epistemic reliability indicators, and policy priority weights reflecting governance requirements and user preferences. Candidate responses are ranked according to their composite utility scores, with higher scores indicating better overall quality, lower conflict, lower uncertainty, and stronger policy alignment. The system selects the highest-ranked candidate response that satisfies all mandatory governance constraints, does not exceed conflict or uncertainty thresholds that would trigger safety-mode degradation, and meets minimum quality standards across all critical framework evaluators. If no candidate response satisfies these criteria, the system may generate a conservative fallback response, request additional user clarification, or escalate to human review depending on the deployment configuration and severity of the constraint violations or uncertainty issues.

### Output Generation

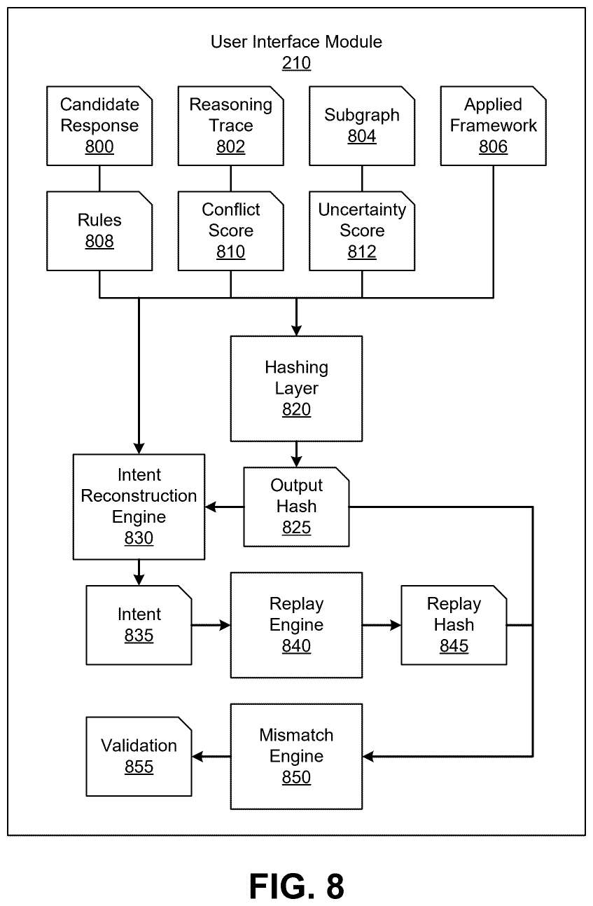

FIG. 8 illustrates a user interface module 210 that processes candidate responses 800, reasoning traces 802, subgraphs 804, applied frameworks 806, rules 808, conflict scores 810, and uncertainty scores 812 through a hashing layer 820 to generate an output hash 825, which is used by an intent reconstruction engine 830, replay engine 840, and mismatch engine 850 to produce validation results 855. The user interface module 210 receives the selected candidate response along with all associated reasoning artifacts generated during the evaluation and selection process. The hashing layer 820 applies cryptographic hashing functions to these components to generate a tamper-evident output hash 825 that uniquely identifies the reasoning state and enables deterministic reconstruction. The intent reconstruction engine 830 uses the output hash 825 and stored audit metadata to reconstruct the original intent representation that initiated the reasoning process, enabling verification that the output appropriately addresses the user's query. The replay engine 840 can re-execute the reasoning process using the reconstructed intent 835 to generate a replay hash 845, and the mismatch engine 850 compares the original output hash 825 with the replay hash 845 to detect any discrepancies that might indicate system changes, data corruption, or non-deterministic behavior. The validation results 855 indicate whether the reasoning process is reproducible and whether the output packet maintains integrity.

The system generates a structured output packet, comprising a validated response, a machine-readable explanation trace, provenance references to HUKG elements, quantified uncertainty indicators, and safety or escalation flags, where applicable. The validated response contains the natural language answer content selected from the candidate responses after framework evaluation, conflict resolution, and governance enforcement. The machine-readable explanation trace is a structured data object that documents the reasoning process, including the sequence of knowledge graph nodes accessed, the framework evaluators applied, the constraint rules enforced, and the intermediate reasoning steps performed. Provenance references link each claim or assertion in the response back to specific nodes in the HUKG, enabling users or auditors to verify the source of information and assess its reliability. Quantified uncertainty indicators include the uncertainty score and its component metrics, providing explicit disclosure of epistemic limitations and confidence levels. Safety or escalation flags indicate whether the response triggered any governance constraints, whether safety-mode degradation was activated, or whether human review is recommended.

A user interface module 210 receives the candidate response 800, reasoning trace 802, subgraph 804, applied framework 806, rules 808, conflict score 810, and uncertainty score 812. The user interface module 210 serves as the integration point where all reasoning artifacts are collected and packaged for presentation to the user and for audit logging. The candidate response 800 is the selected natural language output that will be displayed to the user. The reasoning trace 802 documents the generation and evaluation process. The subgraph 804 contains the knowledge elements from the HUKG that were referenced during reasoning. The applied framework 806 identifies which framework evaluators were used to assess the response. The rules 808 specify which governance constraints were enforced. The conflict score 810 quantifies cross-framework disagreement. The uncertainty score 812 quantifies overall epistemic reliability. These components are transmitted to the hashing layer 820 for integrity protection and to the audit logging subsystem for persistent storage.

A hashing layer 820 generates an output hash 825 from these components to ensure integrity and enable deterministic reconstruction. The hashing layer 820 applies cryptographic hash functions such as SHA-256 or similar algorithms to compute a fixed-length digest that uniquely represents the complete reasoning state. The hash computation includes all reasoning artifacts to ensure that any modification to the response content, reasoning trace, referenced knowledge, applied frameworks, enforced rules, or computed scores would result in a different hash value. The output hash 825 serves as a tamper-evident seal that enables detection of unauthorized modifications and as a unique identifier that enables retrieval of the complete reasoning context from audit logs. The output hash 825 is stored in the audit record along with timestamps, model version identifiers, and policy version identifiers to enable deterministic reconstruction of the reasoning process under equivalent system conditions.

An intent reconstruction engine 830 uses the output hash 825 to reconstruct the intent 835 that led to the output, enabling verification and audit. The intent reconstruction engine 830 queries the audit logging subsystem using the output hash 825 as a key to retrieve the stored reasoning artifacts, including the original normalized intent representation that initiated the reasoning process. The engine reconstructs the intent 835 by deserializing the stored intent representation and verifying its integrity through hash validation. The reconstructed intent 835 enables auditors or system operators to verify that the output appropriately addresses the user's original query, that the reasoning process followed the expected workflow, and that all governance constraints were properly enforced. The intent reconstruction capability supports regulatory compliance requirements for explainability and auditability in safety-sensitive or regulated deployment environments.

A replay engine 840 can re-execute the reasoning process using the stored intent 835 to generate a replay hash 845, and a mismatch engine 850 compares the original output hash 825 with the replay hash 845 to produce validation results 855. The replay engine 840 re-executes the complete reasoning workflow using the reconstructed intent 835 as input, applying the same model versions, policy versions, and governance rules that were active during the original reasoning session. The replay process generates a new output packet and computes a replay hash 845 from the replayed reasoning artifacts. The mismatch engine 850 compares the original output hash 825 with the replay hash 845 to determine whether the reasoning process is deterministic and reproducible. If the hashes match, the validation results 855 indicate successful reproduction, confirming that the reasoning infrastructure operates deterministically under equivalent conditions. If the hashes differ, the validation results 855 indicate a mismatch, which may trigger investigation into potential sources of non-determinism, system changes, or data corruption.

The system writes a versioned audit record, including identifiers for model versions, policy versions, applied constraint rules, conflict metrics, uncertainty metrics, and cryptographic hashes of the output packet. The audit record is a structured data object stored in persistent, append-only storage that captures the complete reasoning context necessary for reproducibility and compliance verification. Model version identifiers specify which statistical inference models, framework evaluators, and constraint engines were active during reasoning. Policy version identifiers specify which governance rules, safety constraints, and jurisdictional overlays were enforced. Applied constraint rule identifiers enumerate which specific rules were triggered and what enforcement actions were taken. Conflict metrics record the cross-framework disagreement scores computed during evaluation. Uncertainty metrics record the epistemic reliability scores computed during uncertainty propagation. Cryptographic hashes of the output packet provide tamper-evident integrity protection and enable deterministic reconstruction. The audit record enables regulatory inspection, compliance verification, litigation defense, and system debugging by preserving the complete reasoning state in a machine-readable, verifiable format.

### Longitudinal Profiling

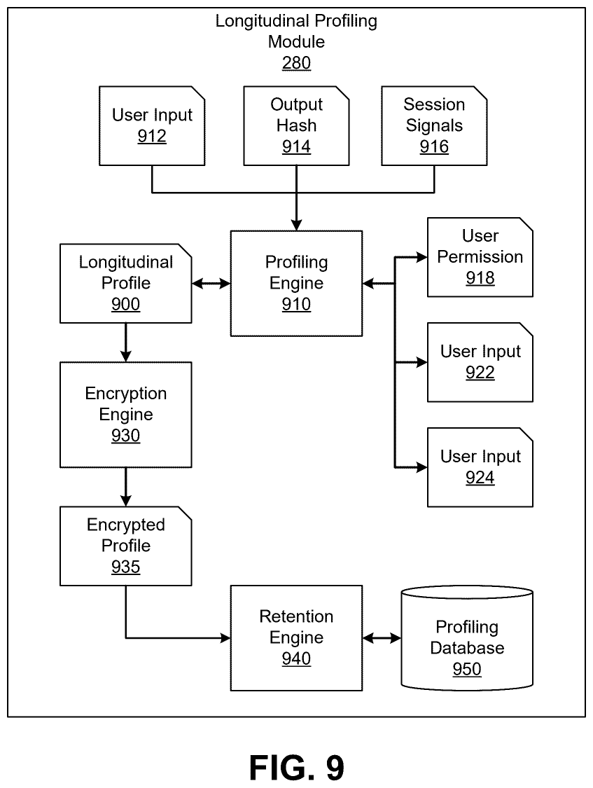

FIG. 9 illustrates a longitudinal profiling module 280 comprising a profiling engine 910 that processes user input 912, output hash 914, and session signals 916 subject to user permission 918 to maintain a longitudinal profile 900. An encryption engine 930 may encrypt the longitudinal profile 900 for storage as an encrypted profile 935 in a profiling database 950. A retention engine 940 may maintain and enforce retention controls.

The longitudinal profiling module 280 enables adaptive personalization of reasoning behavior across multiple sessions while preserving user privacy and maintaining governance supremacy. The profiling engine 910 receives user input 912 from the current session, the output hash 914 identifying the selected response, and session signals 916 capturing interaction patterns and feedback. The profiling engine 910 updates the longitudinal profile 900 only when explicit user permission 918 has been granted, ensuring that personalization occurs under informed consent. The encryption engine 930 applies cryptographic encryption to the longitudinal profile 900 to generate an encrypted profile 935 that protects user privacy during storage and transmission. The encrypted profile 935 is stored in the profiling database 950, and the retention engine 940 enforces user-defined retention and deletion policies to ensure compliance with privacy regulations and user preferences.

The system maintains a longitudinal reasoning profile for each user, subject to explicit consent and retention controls. The longitudinal reasoning profile is a structured data object that accumulates information about user preferences, interaction patterns, and reasoning outcomes across multiple sessions to enable personalized adaptation of the reasoning infrastructure. The profile is maintained only for users who have provided explicit consent for data collection and personalization, with consent mechanisms integrated into the user interface to ensure informed decision-making. Retention controls specify how long profile data is stored, under what conditions it may be accessed or modified, and when it must be automatically deleted. Users may revoke consent at any time, triggering immediate deletion of their profile data. The profile operates under governance supremacy, meaning that personalization preferences cannot override mandatory safety constraints, regulatory requirements, or domain-of-scope limitations encoded in the constraint execution engine.

A profiling engine 910 processes user input 912, output hash 914, and session signals 916 to update the longitudinal profile 900, but only when user permission 918 has been granted. The profiling engine 910 analyzes user input 912 to identify patterns in query formulation, domain interests, and interaction style that inform personalization. The output hash 914 enables the profiling engine to track which responses were selected and presented to the user, providing feedback about reasoning outcomes. Session signals 916 capture behavioral indicators such as interaction duration, follow-up queries, explicit feedback, or engagement metrics that indicate user satisfaction or dissatisfaction with reasoning outputs. The profiling engine applies machine learning algorithms or rule-based logic to extract personalization parameters from these inputs, updating the longitudinal profile 900 with revised framework weighting preferences, explanation depth settings, or domain scope preferences. Profile updates occur only when user permission 918 is active, with permission status checked before each update operation to ensure ongoing consent.

An encryption engine 930 encrypts the longitudinal profile 900 to protect user privacy. The encryption engine 930 applies symmetric or asymmetric cryptographic algorithms to transform the longitudinal profile 900 into an encrypted profile 935 that is computationally infeasible to decrypt without the appropriate decryption key. The encryption protects user privacy by ensuring that profile data cannot be accessed by unauthorized parties, even if the profiling database 950 is compromised or accessed by system administrators without proper authorization. The encryption keys may be managed through secure key management systems, hardware security modules, or user-controlled key derivation mechanisms depending on the deployment environment and privacy requirements. The encrypted profile 935 is transmitted to the profiling database 950 for persistent storage.

The encrypted profile 935 is stored in a profiling database 950, and a retention engine 940 enforces user-defined retention and deletion policies. The profiling database 950 provides persistent storage for encrypted profiles across multiple users, with access controls that restrict profile retrieval to authorized system components and prevent unauthorized disclosure. The retention engine 940 monitors the age and usage patterns of each stored profile, automatically deleting profiles that exceed their retention window or that belong to users who have revoked consent. Retention policies may specify maximum storage durations, conditions for automatic deletion, or triggers for profile archival. The retention engine 940 generates audit logs documenting all profile access, modification, and deletion events to support compliance verification and privacy auditing. Users may request immediate deletion of their profile at any time, triggering the retention engine to purge all associated data from the profiling database 950 and related system components.

The longitudinal profile 900 may include framework weighting preferences, governance constraint preferences, explanation depth settings, and domain scope preferences, and is used to modulate reasoning behavior across sessions while preserving governance supremacy. Framework weighting preferences specify the relative importance of different framework evaluators in output selection, enabling users to prioritize scientific plausibility over ethical considerations or vice versa according to their values. Governance constraint preferences allow users to impose additional restrictions beyond mandatory safety constraints, such as narrower domain-of-scope limitations or stricter content filtering. Explanation depth settings control the level of detail provided in explanation traces, with some users preferring concise summaries and others preferring comprehensive documentation of reasoning steps. Domain scope preferences indicate which subject matter areas or knowledge graph regions are most relevant to the user's typical queries, enabling more efficient knowledge retrieval. These preferences modulate reasoning behavior by influencing prompt generation, framework selection, output formatting, and explanation generation, but they cannot override mandatory governance constraints, safety boundaries, or regulatory requirements enforced by the constraint execution engine.

In one or more embodiments, the longitudinal profiling module 280 receives user feedback to the presentation of the selected response through the user interface module 210, wherein the feedback may comprise explicit ratings, implicit engagement signals, follow-up queries, or behavioral indicators captured during or after response presentation. The profiling engine 910 processes the user feedback in conjunction with the output hash 914 and the reasoning trace associated with the selected response to generate a feedback-based quality score that quantifies the degree of user satisfaction or dissatisfaction with the reasoning output. The quality score may be computed using supervised learning algorithms trained on historical feedback patterns, rule-based scoring functions that map feedback types to numerical values, or hybrid approaches that combine multiple feedback signals into a composite assessment. The profiling engine 910 uses the quality score to modify the longitudinal profile 900 by adjusting framework weighting preferences to favor evaluators that contributed to highly-rated responses, updating explanation depth settings to match user preferences inferred from feedback patterns, or refining domain scope preferences based on topics associated with positive or negative feedback. The profile modifications occur only when user permission 918 remains active and when the feedback signals meet minimum confidence thresholds to prevent profile corruption from spurious or low-quality feedback data.

### Example Methods

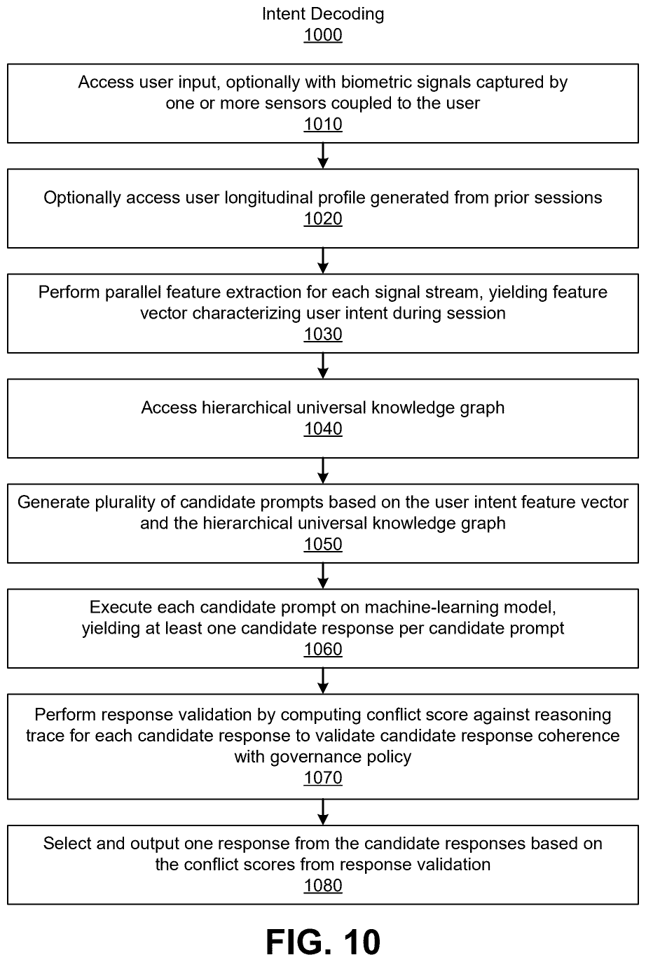

FIG. 10 is a method flowchart describing constraint-governed neuro-symbolic reasoning, according to some embodiments. The process may entail receiving user input, intent decoding, knowledge graph access, candidate prompt generation, machine-learning model execution, response validation with conflict score computation, response selection and output, or some combination thereof.

The system accesses 1010 user input, optionally with biometric signals captured by one or more sensors coupled to the user. The user input may comprise natural language text entered through a text input interface, voice commands captured through a microphone, or other forms of user interaction with the client device. The biometric signals may include heart rate variability, respiratory cadence, electroencephalography signals, galvanic skin response, or other physiological measurements captured by sensors coupled to or integrated with the user device. The system captures these input streams in parallel to enable multimodal intent inference. The captured signals are transmitted to the intent decoding module for feature extraction and cognitive state estimation.

The system optionally accesses 1020 a user longitudinal profile generated from prior sessions. The longitudinal profile comprises framework weighting preferences, governance constraint preferences, explanation depth settings, domain scope preferences, and historical interaction patterns accumulated across previous reasoning sessions. The profile is accessed only when explicit user consent has been granted and when the profile has not exceeded its retention window. The profile data is decrypted and loaded into memory for use in personalizing the current reasoning session. The profile influences prompt generation, framework selection, and output formatting while remaining subordinate to mandatory governance constraints.

The system performs 1030 parallel feature extraction for each signal stream, yielding a feature vector characterizing user intent during the session. Feature extraction operates independently on each input modality, including textual input, biometric signals, and contextual metadata, to generate modality-specific feature representations. The extracted features are processed through a cognitive state estimation engine that infers user objectives, emotional state, attention stability, and domain context. The feature vectors from all modalities are fused into a unified intent representation that encodes the user's query, inferred goals, safety category, and requested output structure. This normalized intent representation serves as the primary input for downstream knowledge retrieval and prompt generation.

The system accesses 1040 the hierarchical universal knowledge graph. The knowledge graph comprises typed nodes representing concepts, claims, frameworks, rules, and sources, interconnected by typed edges encoding relationships such as supports, contradicts, refines, and governed_by. The system queries the graph using the normalized intent representation to identify relevant subgraphs containing knowledge elements pertinent to the user's query. The retrieval process applies policy-filtered constraints to ensure that only knowledge elements consistent with applicable governance rules are included. The retrieved subgraphs include provenance metadata, confidence weights, and framework mappings that enable structured reasoning and uncertainty propagation.

The system generates 1050 a plurality of candidate prompts based on the user intent feature vector and the hierarchical universal knowledge graph. Each candidate prompt is constructed by conditioning on the retrieved knowledge subgraphs and the normalized intent representation to create a structured input for the language model. The prompt generation process incorporates personalization profile constraints, active framework specifications, and governance boundaries to ensure that generated prompts align with user preferences and policy requirements. Multiple candidate prompts may be generated to explore different reasoning pathways or to enable downstream validation through comparison. The candidate prompts are formatted according to the input requirements of the language model and transmitted for execution.

The system executes 1060 each candidate prompt on a machine-learning model, yielding at least one candidate response per candidate prompt. The machine-learning model may be a language model that applies statistical inference over its learned parameter space to generate natural language responses conditioned on the structured prompts. Each execution produces a candidate response along with an associated reasoning trace that documents the intermediate reasoning steps, referenced knowledge elements, and applied inference rules. The language model may generate multiple candidate responses per prompt through sampling or beam search to increase the diversity of outputs available for validation. The candidate responses and reasoning traces are transmitted to the response validation module and governance module for evaluation.

The system performs 1070 response validation by computing a conflict score against a reasoning trace for each candidate response to validate candidate response coherence with governance policy. The response validation module executes multiple independent framework evaluators, each assessing the candidate response according to a distinct reasoning framework such as scientific plausibility, ethical acceptability, policy compliance, or risk assessment. Each evaluator produces a quantitative score, structured rationale, and constraint signals that indicate the degree of alignment between the candidate response and the framework's criteria. The system computes a conflict score by quantifying divergence among the framework evaluator outputs, measuring pairwise score differences, contradiction density within referenced knowledge elements, and rule collision events. The conflict score, along with coverage metrics, variance scores, and rule violation indicators, is aggregated into an uncertainty representation that characterizes the reliability of each candidate response.

The system selects and outputs 1080 one response from the candidate responses based on the conflict scores from response validation. The selection process ranks candidate responses according to a composite utility function that combines framework evaluation scores, conflict-adjusted rankings, uncertainty weightings, governance compliance states, and longitudinal profile preferences. The system selects the candidate response with the highest composite utility score, provided that the response satisfies all hard governance constraints and does not exceed uncertainty or conflict thresholds that would trigger safety-mode degradation. The selected response is packaged into a structured output packet that includes the validated answer content, machine-readable explanation trace, provenance references to knowledge graph elements, quantified uncertainty indicators, and any applicable safety or escalation flags. The output packet is transmitted to the user interface module for presentation to the user and to the audit logging subsystem for versioned record creation.

By embedding governance enforcement, conflict quantification, and uncertainty propagation directly within the reasoning pipeline, the disclosed architecture provides material technical improvements over conventional probabilistic language generation systems, including deterministic governance enforcement rather than post-hoc moderation, structured cross-framework transparency, formalized conflict detection and reconciliation, quantified uncertainty rather than heuristic confidence, machine-readable reasoning artifacts enabling reproducibility, and controlled longitudinal personalization bounded by consent and retention policies.

The result is a constraint-executable reasoning infrastructure suitable for regulated, enterprise, and safety-critical environments requiring traceable, explainable, and auditable artificial intelligence.

### Machine-Learning Configurations

In various embodiments, a wide variety of machine learning techniques may be used. Examples include supervised, unsupervised, and semi-supervised learning such as decision trees, support vector machines (SVMs), regression, Bayesian networks, and genetic algorithms. Deep learning techniques such as neural networks, including convolutional neural networks (CNN), recurrent neural networks (RNN), long short-term memory networks (LSTM), transformers, and linear recurrent neural networks (e.g., Mamba), may also be used. For example, various intent decoding tasks performed by the intent decoding module 220—such as domain classification, entity extraction, inferred objectives, safety category tagging, and requested output structure inference—may apply one or more machine learning and deep learning techniques; multi-framework evaluation and scoring tasks performed by the response validation module 270—such as scientific plausibility, ethical acceptability, policy compliance, risk assessment, score computation, contradiction detection, constraint signal generation, conflict metric computation, and uncertainty aggregation—may apply one or more machine learning and deep learning techniques; and other processes including graph retrieval and subgraph curation by the graph curation module 230, prompt construction by the prompt generation module 240, executable constraint enforcement by the governance module 260, and consent-bound personalization by the longitudinal profiling module 280 may similarly employ machine learning and deep learning approaches.

One or more deep neural networks (DNNs) may be used in various embodiments. A DNN may refer to an AI model that includes at least 5 hidden layers, at least 50 learnable computational nodes in the aggregate across the hidden layers, and at least 2,000 trainable parameters, which may include weights, biases, attention tensors, or other learned coefficients. In some embodiments, the DNN may include between 5 and 16 hidden layers. In some embodiments, the DNN may include between 16 and 64 hidden layers. In some embodiments, the DNN may include between 64 and 256 hidden layers. In some embodiments, the DNN may include between 256 and 1,000 hidden layers. In some embodiments, the DNN may include more than 1,000 layers. In some embodiments, the number of learnable computational nodes may range from 50 to 1,000, from 1,000 to 10,000, from 10,000 to one million, or may exceed one million. In some embodiments, the total number of trainable parameters may range from 2,000 to 10,000, from 10,000 to 100,000, from 100,000 to one million, from one million to ten million, from ten million to a hundred million, from a hundred million to a billion, or may exceed one billion. These embodiments encompass neural networks of varying sizes and complexities, including convolutional networks, recurrent networks, transformer-based architectures, and other variants that meet the minimum structural thresholds described above.

In various embodiments, the training techniques for a machine learning model may be supervised, semi-supervised, or unsupervised. In supervised learning, the machine learning models may be trained with a set of training samples that are labeled. For example, for a machine learning model trained to infer a normalized intent representation, the training samples may be historical user queries paired with contextual signals (e.g., session metadata and optional biometric streams such as heart rate variability, respiratory cadence, EEG, GSR, motion/posture, and interaction telemetry) and ground-truth intent annotations. The labels for each training sample may be binary or multi-class. In training a machine learning model for policy compliance classification, the training labels may include a positive label that indicates policy-compliant content that may be permitted or repaired, and a negative label that indicates policy-violating content that must be blocked, redirected, or escalated. In some embodiments, the training labels may also be multi-class such as domain taxonomy classes, requested output structures (e.g., summary, step-by-step, audit packet), safety category tags, and governance action classes (block/repair/redirect/escalate).

By way of example, the training set may include multiple past records (e.g., session artifacts) with known outcomes. Each training sample in the training set may correspond to a past session, and the corresponding governance decision or evaluator score may serve as the label for the sample. A training sample may be represented as a feature vector that includes multiple dimensions. Each dimension may include data of a feature, which may be a quantized value of an attribute that describes the past record. For example, in a machine learning model that is used to rank and retrieve policy-scoped, provenance-rich subgraphs from the hierarchical universal knowledge graph (HUKG), the features in a feature vector may include entity and concept embeddings, framework mappings indicating applicable evaluators, rule/governance overlays relevant to the query, node and edge confidence weights, contradiction density within the subgraph, jurisdictional tags, provenance trust scores, and retrieval coverage indicators. In various embodiments, certain pre-processing techniques may be used to normalize the values in different dimensions of the feature vector, apply time-aware decay modeling for staleness, and filter features according to policy constraints prior to ranking.

In some embodiments, an unsupervised learning technique may be used. The training samples used for an unsupervised model may also be represented by feature vectors, but may not be labeled. Various unsupervised learning techniques such as clustering may be used to determine similarities among the feature vectors, thereby categorizing the training samples into different clusters. For example, clustering of intent embeddings may be used to discover common query archetypes and safety cohorts, and graph-embedding clustering may be used to group semantically related concept/claim nodes and detect contradiction hotspots. In some cases, the training may be semi-supervised with a training set having a mix of labeled samples and unlabeled samples.

A machine learning model may be associated with an objective function, which generates a metric value that describes the objective goal of the training process. The training process may intend to reduce the error rate of the model in generating predictions. In such a case, the objective function may monitor the error rate of the machine learning model. In a model that generates predictions, the objective function of the machine learning algorithm may be the training error rate when the predictions are compared to the actual labels. Such an objective function may be called a loss function. Other forms of objective functions may also be used, particularly for unsupervised learning models whose error rates are not easily determined due to the lack of labels. In some embodiments, in evaluator-score prediction, the objective function may correspond to mean squared error between predicted framework scores and ground-truth evaluator scores; in policy compliance classification, the objective function may correspond to cross-entropy loss over compliant vs. violation labels derived from governance outcomes; and in retrieval embedding learning, the objective function may correspond to contrastive or triplet loss to maximize similarity between intent embeddings and relevant HUKG subgraphs while separating non-relevant subgraphs. In various embodiments, the error rate may be measured as cross-entropy loss, L1 loss (e.g., the sum of absolute differences between the predicted values and the actual value), and L2 loss (e.g., the sum of squared distances).

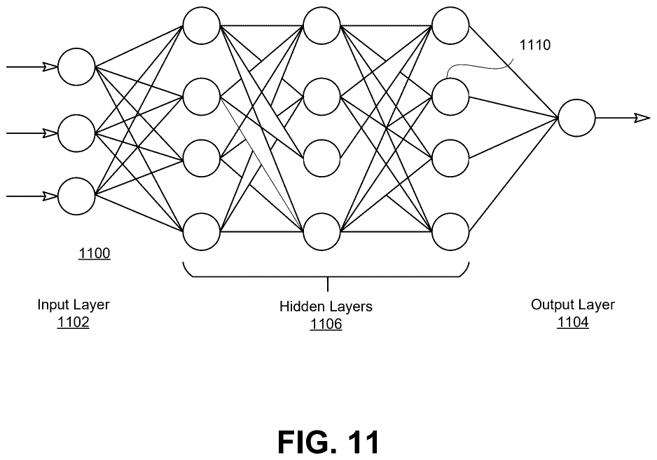

Referring to FIG. 11, a structure of an example neural network is illustrated, in accordance with some embodiments. The neural network 1100 may receive an input and generate an output. The input may be the feature vector of a training sample in the training process and the feature vector of an actual case when the neural network is making an inference. The output may be the prediction, classification, or another determination performed by the neural network. The neural network 1100 may include different kinds of layers, such as convolutional layers, pooling layers, recurrent layers, fully connected layers, and custom layers. A convolutional layer convolves the input of the layer (e.g., an image or tensor) with one or more kernels to generate feature maps. Each convolution result may be associated with an activation function. A convolutional layer may be followed by a pooling layer that selects the maximum value (max pooling) or average value (average pooling) from the portion of the input covered by the kernel size, thereby reducing spatial size. In some embodiments, a pair of convolutional layer and pooling layer may be followed by a recurrent layer that includes one or more feedback loops to account for spatial or temporal relationships. The layers may be followed by multiple fully connected layers used for classification, regression, or object detection. In one embodiment, one or more custom layers may also be present for the generation of a specific format of the output. For example, a custom layer may be used for segmentation or structured token alignment for explanation trace tagging.

The order of layers and the number of layers of the neural network 1100 may vary in different embodiments. In various embodiments, a neural network 1100 includes one or more layers 1102, 1104, and 1106, but may or may not include any pooling layer or recurrent layer. If a pooling layer is present, not all convolutional layers are always followed by a pooling layer. A recurrent layer may also be positioned differently at other locations of the CNN or hybrid architecture. For each convolutional layer, the sizes of kernels (e.g., 3×3, 5×5, 7×7, etc.) and the numbers of kernels allowed to be learned may be different from other convolutional layers.

A machine learning model may include certain layers, nodes 1110, kernels and/or coefficients. Training of a neural network, such as the NN 1100, may include forward propagation and backpropagation. Each layer in a neural network may include one or more nodes, which may be fully or partially connected to other nodes in adjacent layers. In forward propagation, the neural network performs computation in the forward direction based on the outputs of a preceding layer. The operation of a node may be defined by one or more functions. The functions that define the operation of a node may include various computation operations such as convolution with one or more kernels, pooling, recurrent loops in RNNs, gates in LSTMs, attention operations in transformers, etc. The functions may also include an activation function that adjusts the weight of the output of the node. Nodes in different layers may be associated with different functions.

Training of a machine learning model may include an iterative process that includes iterations of making determinations, monitoring the performance of the machine learning model using the objective function, and backpropagation to adjust the weights (e.g., weights, kernel values, coefficients) in various nodes 1110. Training may include initial training, fine tuning, pre-training, post-training, and other different stages of training. A computing device may receive a training set that includes versioned historical reasoning sessions comprising user queries, retrieved HUKG subgraphs (with typed nodes/edges and metadata), reasoning traces, framework evaluator outputs (scores and rationales), governance compliance indicators (constraint signals and actions), and computed conflict/uncertainty metrics. Each training sample in the training set may be assigned with labels indicating intent domain class, requested output structure, safety category, compliance state (e.g., block/repair/redirect/escalate), and, where applicable, target evaluator scores. The computing device, in a forward propagation, may use the machine learning model to generate predicted intent classifications, subgraph relevance scores, compliance decisions, and/or framework score predictions. The computing device may compare the predicted outcomes with the labels of the training sample. The computing device may adjust, in a backpropagation, the weights of the machine learning model based on the comparison. The computing device backpropagates one or more error terms obtained from one or more loss functions to update a set of parameters of the machine learning model. The backpropagating may be performed through the machine learning model and one or more of the error terms based on a difference between a label in the training sample and the generated predicted value by the machine learning model.

By way of example, each of the functions in the neural network may be associated with different coefficients (e.g., weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After an input is provided into the neural network and passes through the neural network in the forward direction, the results may be compared to the training labels or other values in the training set to determine the neural network's performance. The process of prediction may be repeated for other samples in the training sets to compute the value of the objective function in a particular training round. In turn, the neural network performs backpropagation by using gradient descent such as stochastic gradient descent (SGD), adaptive methods, or second-order methods to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing intent decoding, HUKG subgraph ranking, multi-framework score prediction, policy compliance verification, conflict estimation, uncertainty scoring, validated output selection, and audit-grade packet generation within the analytics system 110 (modules 220, 230, 240, 250, 260, 270, and 280).

In various embodiments, the training samples described above may be refined and continue to re-train the model, improving the model's ability to perform the inference tasks. In some embodiments, this training and re-training process may repeat, resulting in a computer system that continues to improve its functionality through a use-retraining cycle. For example, after the model is trained, multiple rounds of re-training may be performed. The process may include periodically retraining the machine learning model. The periodic retraining may include obtaining an additional set of training data from newly ingested primary sources, updated provenance nodes/edges, session telemetry and explicit feedback, governance outcomes captured in audit records, and graph curation events. Retraining may be triggered by shifts in conflict metrics, elevated uncertainty scores, changes in retrieval coverage, updates to policy/rule version identifiers, model version updates, or knowledge graph state changes. The process may also include applying the additional set of training data to the machine learning model and adjusting parameters based on the application of the additional set of training data. The retraining may be triggered by any retraining criteria discussed in this disclosure, such as a predictability or a loss function value below a threshold.

In some embodiments, model distillation may be used to transfer knowledge from a trained model to another model with reduced complexity while maintaining similar predictive performance. A trained model, often referred to as a teacher model, may generate outputs such as logits, confidence scores, or probability distributions over possible predictions. These outputs may serve as soft labels for training a student model, which typically has fewer parameters or a more efficient architecture. The student model may learn to approximate the teacher model's decision boundary by minimizing the difference between its own predictions and the teacher model's outputs. This process may involve loss functions such as Kullback-Leibler (KL) divergence or cross-entropy loss with temperature scaling to ensure that the student model effectively captures the patterns and representations learned by the teacher model. Distillation supports deployment in cloud, enterprise, on-device, and edge-computing environments where resource constraints or latency requirements apply.

In some embodiments, model distillation may be performed using different techniques depending on the level of knowledge transfer between the teacher and student models. One common approach is logit-based distillation, where the student model learns from the soft probability distributions produced by the teacher model instead of hard class labels, often with temperature scaling to smooth distributions. Another approach is feature-based distillation, where intermediate feature representations from the teacher model are used to guide the student model's learning process by matching layer activations, embeddings, or attention maps. Additionally, response-based distillation may be used, where the teacher model's decisions, such as classification outputs or ranking scores, are directly used to supervise the student model. In some cases, hybrid approaches may combine multiple distillation techniques to optimize both predictive accuracy and model efficiency.

In some embodiments, model distillation may be applied in scenarios where computational efficiency is a priority, such as deploying models on edge devices, mobile applications, or cloud environments with resource constraints. The distilled student model may inherit the generalization ability of the teacher model while reducing memory footprint and inference latency. In some cases, multiple teacher models may be used to provide diverse outputs, allowing the student model to integrate knowledge from different sources (e.g., scientific, policy, risk evaluators). The distillation process may also incorporate additional regularization techniques, such as weight pruning or quantization, to further optimize the student model's efficiency. By leveraging model distillation, a system may achieve a balance between model performance and computational efficiency, enabling scalable deployment across the analytics system 110 while maintaining deterministic governance enforcement, conflict quantification, uncertainty propagation, and audit-grade traceability.

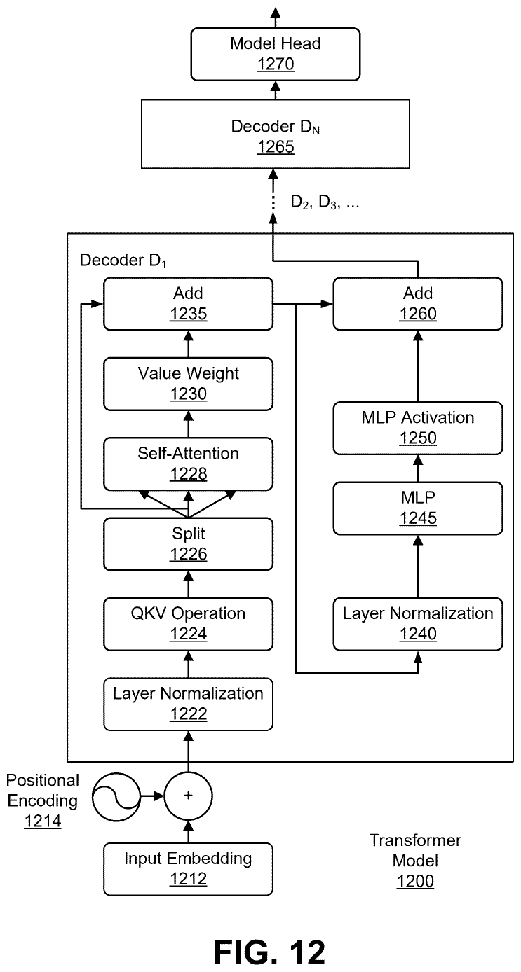

FIG. 12 is a conceptual diagram of functional blocks of a transformer-based neural network model 1200, in accordance with some embodiments. For simplicity, the transformer-based neural network model 1200 is referred to as a transformer model 1200. The transformer model is an example of a machine-learning model discussed in this disclosure. An actual transformer model 1210 may be a large language model that involves numerous neurons, such as a large number of decoders and parameters. The structure illustrated in FIG. 12 is part of a decoder for generating token attention. In a language-processing task related to therapeutic reasoning and intervention generation, the input may take the form of a sequence of words representing a structured prompt encoding multimodal state features and graph context. Each token represents a respective embedding in a latent space. Based on the input tokens, the transformer model 1200 repeatedly generates a sequence of output tokens in an autoregressive manner that correspond to candidate therapeutic actions or interpretive rationales for clinical prompts.

In some embodiments, a transformer model 1200 includes a set of N decoders, D1, D2, . . . DN. Each decoder receives input representations and generates output representations. For example, the first decoder D1 generates intermediate embeddings contextualized for patient psychological state variables and multimodal cues. Each subsequent decoder refines these embeddings using prior decoder outputs and the state-graph context until a final therapeutic recommendation vector is produced. Some decoders may correspond to analyzing data dimensions that model text, audio, video, and physiological indicators. These multimodal streams are used to perform feature integration and therapeutic inference, enabling the model to reason across linguistic, affective, and behavioral data.

The transformer model 1200 may include a model head block 12120 that receives the set of output representations from the final decoder DN and generates an output token as the output for the current iteration. This output may represent natural-language therapeutic guidance, clinician-facing documentation text, or intervention rationale according to safety and regulatory policies managed by the analytics system.

As shown in FIG. 12, a decoder in the transformer model 1200 includes a first layer-normalization block 1222, a query-key-value (QKV) operation block 1224, a split block 1226, a self-attention block 1228, a value-weight block 1230, a first add block 1235, a second layer-normalization block 1240, multi-layer perceptron (MLP) block 1245, an MLP activation block 1250, and a second add block 1260. The operations in the first decoder D1 are exemplary; subsequent decoders may include similar operations. These layers allow the model to attend dynamically to relevant features in multimodal inputs and to latent state-graph variables describing the patient's historical therapeutic context.

FIG. 12 illustrates a flow for the attention mechanism of a transformer model 1200. The transformer model 1200 receives an input sequence such as encoded state-graph data and multimodal embeddings collected from patient and clinician-facing interfaces. Each symbol is converted into a token that takes the form of an embedding vector. The sequence of symbols is represented as a matrix of embedding vectors, each embedding arranged in a row of the matrix. The layer-normalization block 1222 receives the matrix and normalizes its values to stabilize input variance across sessions and modalities.

During training, the transformer model 1200 may be trained in an autoregressive manner using masked label prediction. The input may be a therapeutic prompt sequence encoding prior session data and partially masked outcome labels. To simulate intervention prediction, the system applies masking where unknown intervention types or outcomes are hidden. The decoder attends only to previously observed state nodes and validated interventions while predicting masked positions corresponding to outcome nodes. The objective minimizes prediction error between masked positions and true therapeutic identifiers, enabling the transformer to model long-range dependencies and infer causal relationships between patient states and therapy outcomes.

The QKV operation block 1224 receives the normalized dataset and performs projections to generate query, key, and value matrices. The operation applies learned weights to align representations with contextual signals derived from the longitudinal clinical memory graph (LCMG). The QKV operation models relationships among psychological variables, intervention history, and multimodal affect markers to produce attention distributions guiding therapeutic reasoning and content generation.

The split block 1226 splits the QKV output into query, key, and value matrices. The self-attention block 1228 uses these matrices to generate an attention matrix, applying softmax scaling. The softmax converts logit scores into attention probabilities indicating relevance between patient states and proposed interventions. This attention function allows the transformer model 1200 to associate multimodal patterns with therapy outcomes and prioritize nodes with higher causal relevance within the longitudinal graph.

The value-weight block 1230 receives the attention-score data to generate an attention dataset representing weighted combinations of value vectors. The results are concatenated in the add block 1235 and further normalized by 1240. These operations refine the interpretive context and produce latent embeddings that encode therapeutic rationale, patient progress indicators, and confidence metrics for graph updates.

Each decoder may include one or more MLP blocks 1245 and MLP activation blocks 1250 configured with nonlinear activation functions. The activation functions introduce non-linearity and support mapping of complex psychological transitions. Common activations may include ReLU, tanh, sigmoid, or GeLU. The MLP layers perform feature extraction across multimodal signals, generate compact context embeddings, and select token sequences for subsequent decoding related to therapy planning. Outputs are concatenated by 1260 to complete the cross-modal fusion pipeline.

The output of the first decoder D1 is passed to subsequent decoders until final output data are generated. Each decoder may operate with different trained parameters focused on progressively refined aspects of patient mental-state modeling or intervention inference. The model head block 12120 receives the final output from DN and determines an output token that forms natural-language recommendations or log entries. A softmax operation performed at the LM head selects the next token, resulting in governed therapeutic language or clinician summary text integrated into longitudinal documentation of patient progress.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A computer-implemented method comprising:
accessing user input, provided via an input device on a client device and biometric signals captured by one or more health sensors;
performing parallel feature extraction on the user input and the biometric signals to extract features for each signal stream, wherein the features are concatenated into a feature vector representing intent of the user;
accessing a hierarchical universal knowledge graph comprising a first plurality of nodes representing source material, a second plurality of nodes representing concepts, claims, frameworks, or rules derived from the source material, and a plurality of edges weighting connections between nodes;
generating a plurality of candidate prompts based on the feature vector of the intent of the user and the hierarchical universal knowledge graph;
executing each candidate prompt on a language model to output a plurality of candidate responses to the candidate prompts, wherein the language model is further configured to output a reasoning trace for each candidate response tracing contribution of a subset of nodes in the hierarchical universal graph;
performing validation of each candidate response by computing one or more evaluation scores based on the candidate response and the reasoning trace;
selecting one of the candidate responses based on the evaluation scores; and
transmitting the selected response to the client device for presentation on an output device of the client device.

2. The computer-implemented method of claim 1, wherein accessing the hierarchical universal knowledge graph comprises:
retrieving a structured subgraph responsive to the feature vector, wherein the structured subgraph comprises a subset of the first plurality of nodes and the second plurality of nodes connected by a subset of the plurality of edges.

3. The computer-implemented method of claim 1, wherein the biometric signals comprise one or more of:
heart rate variability signals,
respiratory cadence signals,
electroencephalography signals,
galvanic skin response signals,
motion signals,
posture signals, or
interaction telemetry signals.

4. The computer-implemented method of claim 1, wherein performing parallel feature extraction comprises:
applying a first feature extractor to the user input to extract a first set of features;
applying a second feature extractor to the biometric signals to generate a second set of features; and
concatenating the first set of features and the second set of features to generate the feature vector representing the intent of the user.

5. The computer-implemented method of claim 1, wherein the second plurality of nodes comprises framework nodes representing reasoning frameworks, and wherein performing validation comprises applying a plurality of framework evaluators corresponding to the framework nodes to generate the one or more evaluation scores.

6. The computer-implemented method of claim 5, wherein performing validation further comprises:
computing a conflict score quantifying divergence among the one or more evaluation scores generated by different framework evaluators; and
computing an uncertainty score based on retrieval coverage, the conflict score, weighted rule violations, or model variance, or some combination thereof.

7. The computer-implemented method of claim 1, further comprising:
applying a constraint execution engine to each candidate response to enforce governance rules expressed as executable condition-action logic; and
performing a remedial action to one or more of the candidate responses based on the governance rules.

8. The computer-implemented method of claim 1, further comprising:
generating a structured output packet comprising the selected response and a machine-readable explanation trace linking portions of the selected response to nodes of the hierarchical universal knowledge graph,
wherein transmitting the selected response comprises transmitting the structured output packet to the client device for presentation of the selected response.

9. The computer-implemented method of claim 1, further comprising:
accessing a longitudinal reasoning profile associated with the user, wherein the longitudinal reasoning profile comprises framework weighting preferences, governance constraint preferences, explanation depth settings, retention configuration, or deletion controls, or some combination thereof; and
modulating generation of the plurality of candidate prompts based on the longitudinal reasoning profile.

10. The computer-implemented method of claim 9, further comprising:
receiving user feedback to presentation of the selected response;
scoring the selected response and the reasoning trace based on the user feedback; and
modifying the longitudinal reasoning profile based on the score.

11. A non-transitory computer-readable storage medium storing instructions that, when executed, cause a computer processor to perform operations comprising:
accessing user input, provided via an input device on a client device and biometric signals captured by one or more health sensors;
performing parallel feature extraction on the user input and the biometric signals to extract features for each signal stream, wherein the features are concatenated into a feature vector representing intent of the user;
accessing a hierarchical universal knowledge graph comprising a first plurality of nodes representing source material, a second plurality of nodes representing concepts, claims, frameworks, or rules derived from the source material, and a plurality of edges weighting connections between nodes;
generating a plurality of candidate prompts based on the feature vector of the intent of the user and the hierarchical universal knowledge graph;
executing each candidate prompt on a language model to output a plurality of candidate responses to the candidate prompts, wherein the language model is further configured to output a reasoning trace for each candidate response tracing contribution of a subset of nodes in the hierarchical universal graph;
performing validation of each candidate response by computing one or more evaluation scores based on the candidate response and the reasoning trace;
selecting one of the candidate responses based on the evaluation scores; and
transmitting the selected response to the client device for presentation on an output device of the client device.

12. The non-transitory computer-readable storage medium of claim 11, wherein accessing the hierarchical universal knowledge graph comprises:
retrieving a structured subgraph responsive to the feature vector, wherein the structured subgraph comprises a subset of the first plurality of nodes and the second plurality of nodes connected by a subset of the plurality of edges.

13. The non-transitory computer-readable storage medium of claim 11, wherein the biometric signals comprise one or more of:
heart rate variability signals,
respiratory cadence signals,
electroencephalography signals,
galvanic skin response signals,
motion signals,
posture signals, or
interaction telemetry signals.

14. The non-transitory computer-readable storage medium of claim 11, wherein performing parallel feature extraction comprises:
applying a first feature extractor to the user input to extract a first set of features;
applying a second feature extractor to the biometric signals to generate a second set of features; and
concatenating the first set of features and the second set of features to generate the feature vector representing the intent of the user.

15. The non-transitory computer-readable storage medium of claim 11, wherein the second plurality of nodes comprises framework nodes representing reasoning frameworks, and wherein performing validation comprises applying a plurality of framework evaluators corresponding to the framework nodes to generate the one or more evaluation scores.

16. The non-transitory computer-readable storage medium of claim 15, wherein performing validation further comprises:
computing a conflict score quantifying divergence among the one or more evaluation scores generated by different framework evaluators; and
computing an uncertainty score based on retrieval coverage, the conflict score, weighted rule violations, or model variance, or some combination thereof.

17. The non-transitory computer-readable storage medium of claim 11, the operations further comprising:
applying a constraint execution engine to each candidate response to enforce governance rules expressed as executable condition-action logic; and
performing a remedial action to one or more of the candidate responses based on the governance rules.

18. The non-transitory computer-readable storage medium of claim 11, the operations further comprising:
generating a structured output packet comprising the selected response and a machine-readable explanation trace linking portions of the selected response to nodes of the hierarchical universal knowledge graph,
wherein transmitting the selected response comprises transmitting the structured output packet to the client device for presentation of the selected response.

19. The non-transitory computer-readable storage medium of claim 11, the operations further comprising:
accessing a longitudinal reasoning profile associated with the user, wherein the longitudinal reasoning profile comprises framework weighting preferences, governance constraint preferences, explanation depth settings, retention configuration, or deletion controls, or some combination thereof; and
modulating generation of the plurality of candidate prompts based on the longitudinal reasoning profile.

20. The non-transitory computer-readable storage medium of claim 19, the operations further comprising:
receiving user feedback to presentation of the selected response;
scoring the selected response and the reasoning trace based on the user feedback; and
modifying the longitudinal reasoning profile based on the score.

21. A system comprising:
a computer processor; and
a non-transitory computer-readable storage medium storing instructions that, when executed by the computer processor, cause the computer processor to perform operations comprising:
accessing user input, provided via an input device on a client device and biometric signals captured by one or more health sensors;
performing parallel feature extraction on the user input and the biometric signals to extract features for each signal stream, wherein the features are concatenated into a feature vector representing intent of the user;
accessing a hierarchical universal knowledge graph comprising a first plurality of nodes representing source material, a second plurality of nodes representing concepts, claims, frameworks, or rules derived from the source material, and a plurality of edges weighting connections between nodes;
generating a plurality of candidate prompts based on the feature vector of the intent of the user and the hierarchical universal knowledge graph;
executing each candidate prompt on a language model to output a plurality of candidate responses to the candidate prompts, wherein the language model is further configured to output a reasoning trace for each candidate response tracing contribution of a subset of nodes in the hierarchical universal graph;
performing validation of each candidate response by computing one or more evaluation scores based on the candidate response and the reasoning trace;
selecting one of the candidate responses based on the evaluation scores; and
transmitting the selected response to the client device for presentation on an output device of the client device.
